(19) United States
(12) Patent Application Publication
(10) Pub. No.: US 2006/0252692 A1
(43) Pub. Date: Nov. 9, 2006

(54) Inhibitors for use in hemostasis

(75) Inventors: Gerald W. Lasser, Lynnwood, WA (US); Paul D. Bishop, Fall City, WA (US); Joachim Fruebis, Redmond, WA (US); Woerner P. Meehan, Sammamish, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(73) Assignee: ZymoGenetics, Inc.

(21) Appl. No.: 11/479,928

(22) Filed: Jun. 29, 2006

Related U.S. Application Data
(63) Continuation of application No. 10/385,015, filed on Mar. 10, 2003.
(60) Provisional application No. 60/426,745, filed on Nov. 15, 2002.
(60) Provisional application No. 60/408,798, filed on Sep. 4, 2002.
(60) Provisional application No. 60/385,405, filed on May 31, 2002.
(60) Provisional application No. 60/363,103, filed on Mar. 8, 2002.

(51) Int. Cl. A61K 38/39

(52) U.S. Cl. 514/12

(57) ABSTRACT

The present invention relates to peptide, polynucleotide and fusion proteins for use as inhibitors in hemostasis. These inhibitors are members of the family of proteins bearing a collagen-like domain and a globular domain. The inhibitors are useful for promoting blood flow in the vasculature by reducing thrombogenic and complement activity. The inhibitors are also useful for pacify collagenous surfaces and modulating wound healing.

The present application is a continuation of U.S. patent application Ser. No. 10/385,015, filed Mar. 10, 2003, which claims the benefit of U.S. Patent Application Ser. No. 60/426,745, filed Nov. 15, 2002, U.S. Patent Application Ser. No. 60/408,798, filed Sep. 4, 2002, U.S. Patent Application Ser. No. 60/385,405, filed May 31, 2002, and U.S. Patent Application Ser. No. 60/363,103, filed Mar. 8, 2002, all of which are herein incorporated by reference.

### TECHNICAL FIELD

The present invention relates generally to peptides and polypeptides useful for regulating hemostasis. In particular, the present invention relates to the polypeptide zsig37 and fragments thereof.

### BACKGROUND OF THE INVENTION

Hemostasis is the process that maintains the flow of blood within the circulatory system. Platelets play an early role in hemostasis by forming a thrombus to temporarily repair the vessel damage. While platelets normally do not interact with the endothelium lining of vessel walls, injury to blood vessels, through accident or during surgical procedures, may disrupt the endothelial cell lining. Depending on the extent of the injury, various subendothelial elements such as collagens, elastic lamina or smooth muscle cells with associated fibrillar collagens will be exposed to the flowing blood.

When the subendothelium is exposed following vessel injury, platelets moving in the local blood flow interact with exposed subendothelium matrix containing collagen and decrease blood flow. Further interaction between receptors on the platelet surface and the exposed collagen layer leads to platelet binding and activation resulting in the arrest of local blood flow. The bound platelets are activated and form aggregates with platelets in the passing blood flow through the formation of fibrinogen-interplatelet bridges (Moroi and Jung, Frontiers in Bioscience 3:719 (1998); Barnes et al., Atherosclerosis XI, Jacotot et al. (Eds.), pages 299-306 (Elsevier Science 1998), and Barnes et al., Curr. Opin. Hematol. 5:314 (1998)).

The hemostatic response is graded and dependent on the degree of injury to the blood vessel, the specific blood vessel constituents exposed and the blood flow conditions in the injured area (Rand et al., Thrombosis and Haemostasis 78:445 (1997)). Exposure of the subendothelium matrix (type VI collagen and von Willebrand factor), such as during mild vascular injury, promotes a low degree of adhesion and aggregation in areas with low blood flow conditions. Injuries that result in a greater degree of vascular trauma and exposure of additional vascular constituents, such as the internal elastic lamina and elastin-associated microfibrils, will stimulate the formation of stronger platelet aggregates. Severe vascular trauma, exposing fibril collagens, provokes a thrombotic platelet response, which protects the victim from excessive loss of blood (Rand et al., Thrombosis and Haemostasis 78:445 (1997)).

Complement factor Clq consists of six copies of three related polypeptides (A, B and C chains), with each polypeptide being about 225 amino acids long with a near amino-terminal collagen domain and a carboxy-terminal globular region. Six triple helical regions are formed by the collagen domains of the six A, six B and six C chains, forming a central region and six stalks. A globular head portion is formed by association of the globular carboxy terminal domain of an A, a B and a C chain. Clq is therefore composed of six globular heads linked via six collagen-like stalks to a central fibril region. Sellar et al., Biochem. J. 274:481 (1991). C1q has been found to stimulate defense mechanisms as well as trigger the generation of toxic oxygen species that can cause tissue damage (Tenner, Behring Inst. Mitt. 93:241 (1993)). C1q binding sites are found on platelets. Additionally, complement and C1q play a role in inflammation. The complement activation is initiated by binding of C1q to immunoglobulins.

Inhibitors of hemostasis would be useful for to increase blood flow following vascular injury and to pacify collagenous surfaces, while inhibitors of C1q and the complement pathway would be useful for anti-inflammatory applications, inhibition of complement activation and thrombotic activity.

### BRIEF SUMMARY OF THE INVENTION

The present invention provides peptides, polypeptides, and fusion proteins suitable as therapeutic compounds and methods for using same.

### BRIEF DESCRIPTION OF THE DRAWINGS

FIG. 1 is a schematic showing the concentration-dependent vasorelaxation response of serotonin-contracted rat aortic sections to zsig37.

FIG. 2A is a cross section of a balloon-injured, atherosclerotic rabbit femoral artery. FIG. 2B is a higher magnification of the intimal layer of the femoral artery as shown in FIG. 2A. FIG. 2C is a cross section of a balloon-injured, atherosclerotic rabbit femoral artery after performing a Foltz type crush injury. The schematic of FIG. 2 shows the effect of 1.0 mg/kg zsig37 on blood flow in an athersclerotic Folts model.

FIG. 3 is a schematic showing template bleeding times in cynomolgus macaques following zsig37 (1.0 and 0.5 mg/kg), 1.0 mg/kg BSA or ReoPro™ (0.25 mg/kg) administration. All animals received low molecular weight heparin (1.0 mg/kg).

FIG. 4 is a schematic showing blood loss from punctured iliac arteries of rabbits. Animals were treated with zsig37 (1 mg/kg iv bolus), vehicle control or Clopidogrel (animals were treated 18 hours prior to surgery, 12 mg/kg, and again 45 minutes before surgery, 12 mg/kg). Five minutes after treatment, a 22-gauge Angiocath catheter was briefly inserted into the iliac artery and removed. The resulting bleeding was stopped using standard gauze or gelfoam plus thrombin. Blood loss was determined weighing the gauze pre and post bleeding.

FIG. 5 is a schematic showing zsig37 dose-dependent inhibition of collagen related protein activation of platelets.

FIG. 6 is a schematic showing zsig37 TNF domain inhibition of collagen-induced platelet aggregation.

### DETAILED DESCRIPTION OF THE INVENTION

1. Overview

Human zsig37 is an adipocyte complement related protein homolog that inhibits collagen-mediated platelet activation and the complement pathway, including C1q (see, for example, Sheppard, U.S. Pat. No. 6,265,544 (2001), and PCT publication No. WO00/48625 (2000)). The zsig37 nucleotide sequence (SEQ ID NO:1) encodes a polypeptide (SEQ ID NO:2) having an amino-terminal signal sequence (amino acid residues 1 to 21 of SEQ ID NO:2, or 1 to 25 of SEQ ID NO:2), an adjacent N-terminal region of non-homology (22 to 98 of SEQ ID NO:2), a truncated collagen domain composed of Gly-Xaa-Xaa or Gly-Xaa-Pro repeats and a carboxy-terminal globular portion (amino acid residues 99 to 140 of SEQ ID NO:2), and a carboxy-terminal globular domain (amino acid residues 141 to 281 of SEQ ID NO:2). In addition, the zsig37 amino acid sequence includes ten beta-strands (amino acid residues 147 to 151, 170 to 172, 178 to 181, 185 to 188, 191 to 203, 207 to 214, 219 to 225, 227 to 238, 244 to 250, and 269 to 274 of SEQ ID NO:2) of a “jelly roll” topology that shows significant structural homology to the Tumor Necrosis Factor family. The zsig37 polynucleotide sequence also contains a long 3′ untranslated region. The zsig37 gene was mapped to human chromosome 17, region 17q25.2. SEQ ID NO:3 provides a degenerate nucleotide sequence that encodes the zsig37 polypeptide.

Analysis of the tissue distribution of zsig37 mRNA showed that expression was highest in heart and placenta, with relatively less intense signals in kidney, ovary, adrenal gland, and skeletal muscle. In situ hybridization was performed with a digoxigenin- or biotin-labeled zsig37 probe. Positive signals were observed in the human aorta, heart, prostate, salivary gland, and testis. The positive-staining cells appeared to be endothelial cells of small diameter vessels in the advantitia surrounding the aorta, mesothelial cells overlying the epicardium, acinar cells of the salivary gland, and scattered mononuclear cells, trophoblasts of the placenta, epithelial cells of the prostate and stratified epithelium of the seminiferous tubules of testis.

The binding of biotinylated zsig37 was detected in both activated and nonactivated monocytes, with an increase in zsig37 binding observed in γ-interferon-treated cells. A slight (approximately 10%) reduction in binding was seen in activated cells only when pretreated with 70 fold excess “cold” zsig37. Increased zsig37 binding in activated monocytes suggests that the up-regulation of a monocyte binding protein for zsig37 by inflammatory cytokines. This could potentially result in zsig37 involvement in monocyte phagocytosis, microbial killing, and cellular cytotoxicity. Following the two days in culture, there are macrophages present in the culture and zsig37 may be binding preferentially to this subset of cells. Zsig37 also bound to a mouse monocyte/macrophage line, RAW 264.7 (ATCC No. CRL-2278), indicating macrophage specificity.

A murine ortholog of the zsig37 has been described by Sheppard, U.S. Pat. No. 6,265,544 (2001). The nucleotide, amino acid, and degenerate nucleotide sequences are provided by SEQ ID NOs:4, 5, and 6, respectively.

The present invention provides the use of zsig37 polypeptides and zsig37 polypeptide fragments as inhibitors of hemostasis and immune functions. Either human or murine zsig37 polypeptides are suitable inhibitors.

Illustrative polypeptide fragments include the collagen-like domain of zsig37 polypeptides, ranging from amino acid 99 (Gly) to amino acid 140 (Arg) of SEQ ID NO:2, a portion of the zsig37 polypeptide containing the collagen-like domain or a portion of the collagen-like domain capable of dimerization or oligomerization. Additional exemplary fragments include the globular domain of zsig37 polypeptides, ranging from amino acid 140 (Arg) or 141 (Cys) to 281 (Pro) of SEQ ID NO:2, a portion of the zsig37 polypeptide containing the globular-like domain or an active portion of the globular-like domain. Another zsig37 polypeptide fragment of the present invention include both the collagen-like domain and the globular domain ranging from amino acid residue 99 (Gly) to 281 (Pro) of SEQ ID NO:2. Yet another zsig37 polypeptide fragment of the present invention comprises, or consists of, amino acid residues 26 to 281 of SEQ ID NO:2. Further zsig37 fragments include the following peptides and polypeptides with reference to SEQ ID NO:2: amino acid residue 72 to amino acid residue 78, amino acid residue 72 to amino acid residue 143, amino acid residue 71 to amino acid residue 80, amino acid residue 71 to amino acid residue 99, amino acid residue 71 to amino acid residue 143, amino acid residue 26 to amino acid residue 99, amino acid residue 26 to amino acid residue 140, amino acid residue 26 to amino acid residue 143, amino acid residue 22 to amino acid residue 99, amino acid residue 22 to amino acid residue 140, amino acid residue 22 to amino acid residue 143, and amino acid residue 1 to amino acid residue 99.

The present invention also provides use of zsig37 fusion proteins. For example, fusion proteins of the present invention encompass an immunoglobulin fragment and a zsig37 peptide or polypeptide, as described above. The immunoglobulin moiety of such a fusion protein described herein comprises at least one constant region of an immunoglobulin. Preferably, the immunoglobulin moiety represents a segment of a human immunoglobulin.

Zsig37 peptides, polypeptides, and fusion proteins can be used to inhibit collagen-mediated platelet activation, and to inhibit complement and C1q. In particular, the present invention provides methods for promoting blood flow within the vasculature of a mammal comprising administering to the mammal a therapeutically effective amount of a zsig37 peptide, polypeptide, or fusion protein. The administration of these molecules can reduce thrombogenic and complement activity within the vasculature.

The present invention also provides methods for reducing thrombogenic and complement activity by inhibition of the complement pathway and inhibition collagen-mediated platelet adhesion, activation, or aggregation. In these methods, a zsig37 peptide, polypeptide, or fusion protein can be administered prior to, during, or following an acute vascular injury in the mammal. An example of an acute vascular injury is injury due to vascular reconstruction. Vascular reconstruction can include angioplasty, coronary artery bypass graft, endarterectomy (e.g., carotid endarterectomy), microvascular repair, or anastomosis of a vascular graft. Vascular injury may also be due to trauma, stroke, or aneurysm.

The present invention also provides methods for pacifying damaged collagenous tissues within a mammal comprising administering to the mammal a therapeutically effective amount of a zsig37 peptide, polypeptide, or fusion protein, in which the zsig37 peptide, polypeptide, or fusion protein renders the damaged collagenous tissue inert towards complement activation, thrombotic activity, or immune activation. As an illustration, collagenous tissues may be damaged due to injury associated with ischemia and reperfusion. Within another embodiment, the injury comprises trauma injury ischemia, intestinal strangulation, or injury associated with pre- and post-establishment of blood flow. Within yet another embodiment, the polypeptide is administered to a mammal suffering from cardiopulmonary bypass ischemia and resuscitation, myocardial infarction, or post-trauma vasospasm. Within a related embodiment, the post-trauma vasospasm comprises stroke, percutanious transluminal angioplasty, endarterectomy, accidental vascular trauma or surgical-induced vascular trauma.

The zsig37 peptides, polypeptides, and fusion proteins described herein can be used to prevent occlusion, or to re-establish arterial blood flow, micro-vascular (arteriolar and capillary) blood flow or patency. For example, the zsig37 peptides, polypeptides, and fusion proteins can be used to treat acute coronary syndrome, unstable angina, acute myocardial infarction, peripheral arterial disease, and stroke. The zsig37 peptides, polypeptides, and fusion proteins described herein can be used to treat thrombocytopenia, thrombotic thrombocytopenia purpura, hemolytic uremia syndrome, trauma (e.g., blunt trauma, head trauma, poly-trauma, etc.), deep vein thrombosis, venous thrombosis, and pulmonary embolisms.

The present invention also provides methods of dissolving a thrombus using a zsig37 peptide, polypeptide, or fusion protein. Administration of such a zsig37 therapeutic agent can dissolve a clot causing acute ischemia (e.g., as seen in myocardial infarction, stroke, and the like), peripheral arterial thrombosis, and venous thrombosis.

The present invention further provides methods of pacifying the surface of a prosthetic biomaterial for use in association with a mammal comprising administering to the mammal a therapeutically effective amount of a zsig37 peptide, polypeptide, or fusion protein, in which the zsig37 peptide, polypeptide, or fusion protein renders the surface of the prosthetic biomaterial inert towards complement activation, thrombotic activity, or immune activation. Within one embodiment, the surface of the prosthetic biomaterial is coated with collagen or collagen fragments, gelatin, fibrin, or fibronectin.

The present invention also provides methods of mediating wound repair within a mammal comprising administering to the mammal a therapeutically effective amount of a zsig37 peptide, polypeptide, or fusion protein, in which the zsig37 peptide, polypeptide, or fusion protein enhances progression in wound healing.

Purified recombinant zsig37 polypeptides were found to form oligomers, including trimers, hexamers, 9mers, and 18mers. These forms were active in in vitro assays (Sheppard et al., PCT Publication No. WO00/48625 (2000)). Therefore, the methods described above include the use of oligomers of zsig37 peptides, zsig37 polypeptides, zsig37 fusion proteins, and mixtures thereof. Such oligomers include trimers, hexamers, 9mers, and 18mers. Hexamers may be formed as homotrimers of zsig37, or as homotri-dimers of zsig37.

The present invention also provides pharmaceutical compositions comprising a mixture of zsig37 oligomers. For example, a pharmaceutical composition can comprise a mixture of trimers and hexamers of a polypeptide that comprises amino acid residues 26 to 281 of SEQ ID NO:2. In particular trimer-hexamer mixtures, the ratio of trimer/hexamer may be in the range of about 1/99, 2/98, 3/97, 4/95, 5/95, 6/94, 7/93, 8/92, 9/91, 10/90, 11/89, 12/88, 13/87, 14/86, 15/85, 16/84, 17/83, 18/82, 19/81, 20/80, 25/75, 30/70, 40/60, 50/50, 60/40, 70/30, 75/25, 80/20, 81/19, 82/18, 83/17, 84/16, 85/15, 86/14, 87/13, 88/12, 89/11, 90/10, 91/9, 92/8, 93/7, 94/6, 95/5, 96/4, 97/3, 98/2, or 99/1.

The following fragments of zsig37 can also be useful for the therapeutic methods described herein: amino acid residues 26 to 107 of SEQ ID NO:2, amino acid residues 22 to 107 of SEQ ID NO:2, and amino acid residues 71 to 107 of SEQ ID NO:2. These polypeptides can be administered as single chains or as oligomers, such as homodimers, homotrimers, or homohexamers. Variants of these polypeptides can also be used as therapeutic compounds in which at least one cysteine residue is replaced by a serine residue.

Therapeutic compositions of the present invention include zsig37 heteromers, such as hexamers, which comprise mixtures of zsig37 amino acid sequences, zacrp3 amino acid sequences (Bishop et al., PCT Publication No. WO00/63377), zacrp5 amino acid sequences (Sheppard et al., PCT Publication No. WO00/73444), and zacrp6 amino acid sequences (Sheppard et al., PCT Publication No. WO00/73446).

Therapeutic compositions can also comprise fragments of zsig37, zacrp3, zacrp5, and zacrp6, such as amino acid resides 71 to 80 of SEQ ID NO:2, the zacrp3 amino acid sequence PDCSKCCHGD (SEQ ID NO:7), the zacrp5 amino acid sequence RPCVHCCRPA (SEQ ID NO:8), and the zacrp6 amino acid sequence SGCQRCCDSE (SEQ ID NO:9). Additional therapeutic compositions can comprise fragments of zsig37, zacrp3, zacrp5, and zacrp6, such as amino acid resides 71 to 140 of SEQ ID NO:2, the zacrp3 amino acid sequence PDCSKCCHGD YSFRGYQGPP GPPGPPGIPG NHGNNGNNGA TGHEGAKGEK GDKGDLGPRG ERGQHGPKGE KGYPG (SEQ ID NO:10), the zacrp5 amino acid sequence RPCVHCCRPA WPPGPYARVS DRDLWRGDLW RGLPRVRPTI NIEILKGEKG EAGVRGRAGR SGKEGPPGAR GLQGRRGQKG QVGPPGAA (SEQ ID NO:11), and the zacrp6 amino acid sequence SGCQRCCDSE DPLDPAHVSS ASSSGRPHAL PEIRPYINIT ILKGDKGDPG PMGLPGYMGR EGPQGEPGPQ GSKGDKGEMG SPG (SEQ ID NO:12). These therapeutic compounds can be homomers or heteromers. Illustrative oligomers include homo- and hetero-trimers, as well as homo- and hetero-hexamers.

These and other aspects of the invention will become evident upon reference to the following detailed description. In addition, various references are identified below and are incorporated by reference in their entirety.

2. Definitions

In the description that follows, a number of terms are used extensively. The following definitions are provided to facilitate understanding of the invention.

Unless otherwise specified, “a,” “an,” “the,” and “at least one” are used interchangeably and mean one or more than one.

The term “affinity tag” is used herein to denote a peptide segment that can be attached to a polypeptide to provide for purification or detection of the polypeptide or provide sites for attachment of the polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), substance P, FLAG peptide (Hopp et al., Biotechnology 6:1204 (1988)), streptavidin binding peptide, or other antigenic epitope or binding domain. See, in general Ford et al., Protein Expression and Purification 2:95 (1991). DNAs encoding affinity tags are available from commercial suppliers (e.g., Pharmacia Biotech; Piscataway, N.J.).

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “isolated,” when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774 (1985)).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “ortholog” denotes a polypeptide or protein obtained from one species that is the functional counterpart of a polypeptide or protein from a different species. Sequence differences among orthologs are the result of speciation.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nucleotides in length.

A “polypeptide” is a polymer of amino acid residues joined by peptide bonds, whether produced naturally or synthetically. Polypeptides of less than about 10 amino acid residues are commonly referred to as “peptides.”

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nucleotides, more preferably 20-30 nucleotides. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

3. Production of Nucleic Acid Molecules Encoding Zsig37 Peptides, Polypeptides, and Fusion Proteins

SEQ ID NOs:2 and 4 provide the nucleotide sequences of human zsig37 and murine zsig37, respectively. Nucleic acid molecules encoding human or murine zsig37 polypeptides can be obtained by screening human cDNA or genomic libraries using polynucleotide probes based upon these sequences. Cloning techniques are standard and well-established (see, for example, Ausubel et al. (eds.), Short Protocols in Molecular Biology, 3^{rd} Edition, pages 4-1 to 4-6 (John Wiley & Sons 1995) (“Ausubel (1995)”); Wu et al., Methods in Gene Biotechnology, pages 33-41 (CRC Press, Inc. 1997) (“Wu (1997)”); Ausubel (1995) at pages 5-1 to 5-6; Wu (1997) at pages 307-327)).

Nucleic acid molecules for constructing zsig37 peptides, polypeptides, and fusion proteins can also be obtained by synthesizing nucleic acid molecules using mutually priming long oligonucleotides and the nucleotide sequences described herein (see, for example, Ausubel (1995) at pages 8-8 to 8-9). Established techniques using the polymerase chain reaction provide the ability to synthesize DNA molecules at least two kilobases in length (Adang et al., Plant Molec. Biol. 21:1131 (1993), Bambot et al., PCR Methods and Applications 2:266 (1993), Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993), and Holowachuk et al., PCR Methods Appl. 4:299 (1995)).

The nucleic acid molecules of the present invention can also be synthesized with “gene machines” using protocols such as the phosphoramidite method. If chemically-synthesized double stranded DNA is required for an application such as the synthesis of a gene or a gene fragment, then each complementary strand is made separately. The production of short genes (60 to 80 base pairs) is technically straightforward and can be accomplished by synthesizing the complementary strands and then annealing them. For the production of longer genes (>300 base pairs), however, special strategies may be required, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633 (1990).

Those skilled in the art will readily recognize that, in view of the degeneracy of the genetic code, many nucleotide sequences can encode the zsig37 amino acid sequences described herein. Degenerate nucleotide sequences that encode human zsig37 and murine zsig37 are provided by SEQ ID NOs:3 and 6, respectively. Table 1 sets forth the one-letter codes used within SEQ ID NOs:3 and 6 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| | | | |
|---|---|---|---|
| Nucleotide | Resolution | Complement | Resolution |

| | | | |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | AlT | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOs:3 and 6, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding an amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NOs:2 and 5. Variant sequences can be readily tested for functionality as described herein.

The present invention also provides isolated zsig37 polypeptides that have a substantially similar sequence identity to the polypeptides of SEQ ID NO:2, or their orthologs. The term “substantially similar sequence identity” is used herein to denote polypeptides comprising at least 70%, at least 80%, at least 90%, at least 95%, at least 96%, at least 97%, at least 98%, at least 99%, or greater than 99% sequence identity to the sequence shown in SEQ ID NO:2, or their orthologs. The present invention also includes polypeptides that comprise an amino acid sequence having at least 70%, at least 80%, at least 90%, at least 95%, at least 96%, at least 97%, at least 98%, at least 99%, or greater than 99% sequence identity to the sequence of amino acid residues 22 to 281 or 26 to 281 of SEQ ID NO:2. The present invention further includes nucleic acid molecules that encode such polypeptides. Methods for determining percent identity are described below.

The present invention also contemplates variant zsig37 nucleic acid molecules that can be identified using two criteria: a determination of the similarity between the encoded polypeptide with the amino acid sequence of SEQ ID NO:2, and/or a hybridization assay, as described above. Such zsig37 variants include nucleic acid molecules: (1) that hybridize with a nucleic acid molecule having the nucleotide sequence of SEQ ID NO:1 (or its complement) under stringent washing conditions, in which the wash stringency is equivalent to 0.5×-2×SSC with 0.1% SDS at 55-65° C; or (2) that encode a polypeptide having at least 70%, at least 80%, at least 90%, at least 95%, at least 96%, at least 97%, at least 98%, at least 99%, or greater than 99% identity to the amino acid sequence of SEQ ID NO:2. Alternatively, zsig37 variants can be characterized as nucleic acid molecules: (1) that hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C; and (2) that encode a polypeptide having at least 70%, at least 80%, at least 90%, at least 95%, at least 96%, at least 97%, at least 98%, at least 99%, or greater than 99% sequence identity to the amino acid sequence of SEQ ID NO:2.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes).

$$\frac{Total number of identical matches}{[ \begin{matrix}length of the longer sequence plus the \\ number of gaps introduced into the longer \\ sequence in order to align the two sequences\end{matrix} ]} \times 100$$

TABLE 3

| A | R | N | D | C | Q | E | G | H | I | L | K | M | F | P | S | T | W | Y | V | |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zsig37. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variant zsig37 polypeptides or polypeptides with substantially similar sequence identity are characterized as having one or more amino acid substitutions, deletions or additions. These changes are preferably of a minor nature, that is conservative amino acid substitutions (as shown in Table 4 below) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. Polypeptides comprising affinity tags can further comprise a proteolytic cleavage site between the zsig37 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Determination of amino acid residues that comprise regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity, secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in zsig37 polypeptides so as to minimize disruption of higher order structure essential to biological activity. For example, where the zsig37 polypeptide comprises one or more helices, changes in amino acid residues will be made so as not to disrupt the helix geometry and other components of the molecule where changes in conformation abate some critical function, for example, binding of the molecule to collagen. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same cysteine pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structurally similarities between proteins and polypeptides (Schaanan et al., Science 257:961-964, 1992).

Those skilled in the art will recognize that hydrophilicity or hydrophobicity will be taken into account when designing modifications in the amino acid sequence of a zsig37 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp. For example, residues tolerant of substitution could include Val, Leu and Ile or the group consisting of Met, Gly, Ser, Ala, Tyr and Trp residues as shown in SEQ ID NO:2.

4. Production of Zsig37 Peptides, Polypeptides, and Fusion Proteins

The polypeptides of the present invention can be produced in recombinant host cells following conventional techniques. To express a zsig37-encoding sequence, a nucleic acid molecule encoding the polypeptide must be operably linked to regulatory sequences that control transcriptional expression in an expression vector and then, introduced into a host cell. In addition to transcriptional regulatory sequences, such as promoters and enhancers, expression vectors can include translational regulatory sequences and a marker gene, which is suitable for selection of cells that carry the expression vector.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, an expression vector may comprise a nucleotide sequence that encodes a zsig37-encoding sequence and a secretory sequence derived from any secreted gene. As an illustration, Sheppard, U.S. Pat. No. 6,265,544 (2001), and Sheppard et al., PCT publication No. WO00/48625 (2000), describe the construction of two zsig37 expression vectors, in which the constructs were designed to express a zsig37 polypeptide having a C-terminal (“zSIG37CEE/pZP9”) or N-terminal (“zSIG37NEE/pZP9”) Glu-Glu tag.

Zsig37 peptides, polypeptides, and fusion proteins of the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548), SV40-transformed monkey kidney cells (COS-1; ATCC CRL 1650), and murine embryonic cells (NIH-3T3; ATCC CRL 1658). Sheppard, U.S. Pat. No. 6,265,544 (2001), and Sheppard et al., PCT publication No. WO00/48625 (2000), describe the use of BHK 570 cells to produce zsig37 polypeptides in both small scale and large scale expression systems.

For a mammalian host, the transcriptional and translational regulatory signals may be derived from viral sources, such as adenovirus, bovine papilloma virus, simian virus, and the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Nat'l Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)). One useful combination of a promoter and enhancer is provided by a myeloproliferative sarcoma virus promoter and a human cytomegalovirus enhancer.

Alternatively, a prokaryotic promoter, such as the bacteriophage T3 RNA polymerase promoter, can be used to control production of a zsig37 peptide, polypeptide, or fusion protein in mammalian cells if the prokaryotic promoter is regulated by a eukaryotic promoter (Zhou et al., Mol. Cell. Biol. 10:4529 (1990), and Kaufman et al., Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Zsig37 peptides, polypeptides, and fusion proteins can also be produced by cultured mammalian cells using a viral delivery system. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161 (1994), and Douglas and Curiel, Science & Medicine 4:44 (1997)). Advantages of the adenovirus system include the accommodation of relatively large DNA inserts, the ability to grow to high-titer, the ability to infect a broad range of mammalian cell types, and flexibility that allows use with a large number of available vectors containing different promoters.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. An option is to delete the essential E1 gene from the viral vector, which results in the inability to replicate unless the E1 gene is provided by the host cell. Adenovirus vector-infected human 293 cells (ATCC Nos. CRL-1573, 45504, 45505), for example, can be grown as adherent cells or in suspension culture at relatively high cell density to produce significant amounts of protein (see Garnier et al., Cytotechnol. 15:145 (1994)).

Zsig37 peptides, polypeptides, and fusion proteins can also be expressed in other higher eukaryotic cells, such as avian, fungal, insect, yeast, or plant cells. The baculovirus system provides an efficient means to introduce cloned genes into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the desired polypeptide into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C— or N-terminus of the expressed zsig37 peptide, polypeptide, or fusion protein, for example, a Glu-Glu epitope tag (Grussenmeyer et al., Proc. Nat'l Acad. Sci. 82:7952 (1985)). Using a technique known in the art, a transfer vector containing a nucleotide sequence that encodes a zsig37 peptide, polypeptide, or fusion protein is transformed into E. coli, and screened for bacmids, which contain an interrupted lacZ gene indicative of recombinant baculovirus. The bacmid DNA containing the recombinant baculovirus genome is then isolated using common techniques.

The illustrative PFASTBAC vector can be modified to a considerable degree. For example, the polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins (see, for example, Hill-Perkins and Possee, J. Gen. Virol. 71:971 (1990), Bonning, et al., J. Gen. Virol. 75:1551 (1994), and Chazenbalk and Rapoport, J. Biol. Chem. 270:1543 (1995). In such transfer vector constructs, a short or long version of the basic protein promoter can be used. Moreover, transfer vectors can be constructed, with secretory signal sequences derived from insect proteins. For example, a secretory signal sequence from Ecdysteroid Glucosyltransferase (EGT), honey bee Melittin (Invitrogen Corporation; Carlsbad, Calif.), or baculovirus gp67 (PharMingen: San Diego, Calif.) can be used in such constructs.

The recombinant virus or bacmid is used to transfect host cells. Suitable insect host cells include cell lines derived from IPLB-Sf-21, a Spodoptera frugiperda pupal ovarian cell line, such as Sf9 (ATCC CRL 1711), Sf21AE, and Sf21 (Invitrogen Corporation; San Diego, Calif.), as well as Drosophila Schneider-2 cells, and the HIGH FIVEO cell line (Invitrogen) derived from Trichoplusia ni (U.S. Pat. No. 5,300,435). Commercially available serum-free media can be used to grow and to maintain the cells. Suitable media are Sf900 II™ (Life Technologies) or ESF 921™ (Expression Systems) for the Sf9 cells; and Ex-cellO405™ (JRH Biosciences, Lenexa, KS) or Express FiveO™ (Life Technologies) for the T. ni cells. When recombinant virus is used, the cells are typically grown up from an inoculation density of approximately 2-5×10⁵ cells to a density of 1-2×10⁶ cells at which time a recombinant viral stock is added at a multiplicity of infection (MOI) of 0.1 to 10, more typically near 3.

Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in International Publication Nos. WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, host cells can be used in which both methanol utilization genes (AUG1 and AUG2) are deleted. For production of secreted proteins, host cells can be deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67-88 (CRC Press, 1993).

Alternatively, a zsig37 peptide, polypeptide, or fusion protein can be produced in prokaryotic host cells. Suitable promoters that can be used to produce such amino acid sequences in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH4I, DH5, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)).

When expressing a zsig37 peptide, polypeptide, or fusion protein in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et al. (eds.), page 101 (John Wiley & Sons, Inc. 1996)).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

As an alternative, polypeptides of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)).

5. Assays for Zsig37 Peptides, Polypeptides, and Fusion Proteins

The activity of zsig37 peptides, polypeptides, and fusion proteins on hemostasis, and in particular platelet adhesion and activation leading to platelet aggregation, can be determined using methods and assays provided herein and assays known in the art. Illustrative assays are provided by the Examples.

Collagen is a potent inducer of platelet aggregation, which poses risks to patients recovering from vascular injures. Inhibitors of collagen-induced platelet aggregation would be useful for such purposes. Zsig37 binds to fibronectin and type I, II, III, V and VI collagens. In particular, zsig37 binds to specific domains on collagen VI in a concentration dependent manner. Zsig37 also inhibits collagen-mediated platelet activation. Therefore, zsig37 peptides, polypeptides, and fusion proteins can be used to block the binding of platelets to collagen-coated surfaces, and to reduce associated collagen-induced platelet aggregation.

C1q is a component of the complement pathway and has been found to stimulate defense mechanisms, and to trigger the generation of toxic oxygen species that can cause tissue damage (Tenner, Behring Inst. Mitt. 93:241 (1993)). C1q binding sites are found on platelets. C1q, independent of an immune binding partner, has been found to inhibit platelet aggregation but not platelet adhesion or shape change. The amino terminal region of C1q shares homology with collagen (Peerschke and Ghebrehiwet, J. Immunol. 145:2984 (1990)). Zsig37 binds to complement C1q in a concentration dependent manner, and zsig37 is effective in inhibiting the complement pathway including C1q with both sensitized and unsensitized sheep erythrocytes. These assays can be used to test zsig37 peptides, polypeptides, and fusion proteins.

Zsig37 induces vasodilatation in norepinepherin-contracted aortic rings using the procedures of Dainty et al., J. Pharmacol. 100:767 (1990), and Rhee et al., Neurotox. 16:179 (1995), as is described below in greater detail. This provides another assay to test the activity of a zsig37 peptide, polypeptide, or fusion protein.

Platelet adhesion, activation and aggregation can be evaluated using methods described herein or known in the art, such as the platelet aggregation assay (Chiang et al., Thrombosis Res. 37:605 (1985)), and platelet adhesion assays (Peerschke and Ghebrehiwet, J. Immunol. 144:221 (1990)). Inhibition of C1q and the complement pathway can be determined using methods disclosed herein or know in the art, such as described in Suba and Csako, J. Immunol. 117:304 (1976). Assays for platelet adhesion to collagen and inhibition of collagen-induced platelet aggregation can be measured using methods described in Keller et al., J. Biol. Chem. 268:5450 (1993); Waxman and Connolly, J. Biol. Chem. 268:5445 (1993); Noeske-Jungblut et al., J. Biol. Chem. 269:5050 (1994), and Deckmyn et al., Blood 85:712 (1995).

Various in vitro and in vivo models are available for assessing the effects of zsig37 peptides, polypeptides, fusion proteins on ischemia and reperfusion injury. See for example, Shandelya et al., Circulation 88:2812 (1993); Weisman et al., Science 249:146 (1991); Buerke et al., Circulation 91:393 (1995); Horstick et al., Circulation 95:701 (1997), and Burke et al., J. Phar. Exp. Therp. 286:429 (1998). An ex vivo hamster platelet aggregation assay is described by Deckmyn et al., Blood 85:712 (1995). Bleeding times in hamsters and baboons can be measured following injection of a zsig37 peptide, polypeptide, or fusion protein using the model described by Deckmyn et al., Blood 85:712 (1995). Changes in platelet adhesion under flow conditions following administration of a zsig37 peptide, polypeptide, or fusion protein can be measured using the method described in Harsfalvi et al., Blood 85:705 (1995).

Zsig37 peptides, polypeptides, and fusion proteins can also be evaluated using methods such as healing of dermal layers in pigs (Lynch et al., Proc. Natl. Acad. Sci. USA 84:7696 (1987)) and full-thickness skin wounds in genetically diabetic mice (Greenhalgh et al., Am. J. Pathol. 136:1235 (1990)).

Other suitable assays of zsig37 peptides, polypeptides, and fusion proteins can be determined by those of skill in the art.

6. Production of Zsig37 Conjugates

The present invention includes chemically modified zsig37 peptides, polypeptides, and fusion proteins, in which a zsig37 peptide, polypeptide, or fusion protein is linked with a polymer. Typically, the polymer is water-soluble so that the zsig37-containing sequence does not precipitate in an aqueous environment, such as a physiological environment. An example of a suitable polymer is one that has been modified to have a single reactive group, such as an active ester for acylation, or an aldehyde for alkylation, In this way, the degree of polymerization can be controlled. An example of a reactive aldehyde is polyethylene glycol propionaldehyde, or mono-(C₁-C₁₀)alkoxy, or aryloxy derivatives thereof (see, for example, Harris, et al., U.S. Pat. No. 5,252,714). The polymer may be branched or unbranched. Moreover, a mixture of polymers can be used to produce conjugates of zsig37 peptides, polypeptides, and fusion proteins.

Zsig37-containing conjugates used for therapy can comprise pharmaceutically acceptable water-soluble polymer moieties. Suitable water-soluble polymers include polyethylene glycol (PEG), monomethoxy-PEG, mono-(C₁-C₁₀)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000, and 25,000. A zsig37 conjugate can also comprise a mixture of such water-soluble polymers.

One example of a zsig37-containing conjugate comprises a zsig37 polypeptide moiety and a polyalkyl oxide moiety attached to the N-terminus of the zsig37 peptide, polypeptide, or fusion protein. PEG is one suitable polyalkyl oxide. As an illustration, a zsig37 polypeptide can be modified with PEG, a process known as “PEGylation.” PEGylation of a zsig37 peptide, polypeptide, or fusion protein can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). For example, PEGylation can be performed by an acylation reaction or by an alkylation reaction with a reactive polyethylene glycol molecule. In an alternative approach, zsig37 conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with a zsig37 peptide, polypeptide, or fusion protein. An example of an activated PEG ester is PEG esterified to N-hydroxysuccinimide. As used herein, the term “acylation” includes the following types of linkages between a zsig37 peptide, polypeptide, or fusion protein and a water-soluble polymer: amide, carbamate, urethane, and the like. Methods for preparing PEGylated a zsig37 peptide, polypeptide, or fusion protein by acylation will typically comprise the steps of (a) reacting a zsig37 peptide, polypeptide, or fusion protein with PEG (such as a reactive ester of an aldehyde derivative of PEG) under conditions whereby one or more PEG groups attach to the zsig37 peptide, polypeptide, or fusion protein, and (b) obtaining the reaction product(s). Generally, the optimal reaction conditions for acylation reactions will be determined based upon known parameters and desired results. For example, the larger the ratio of PEG:zsig37 moiety, the greater the percentage of polyPEGylated product.

The product of PEGylation by acylation is typically a polyPEGylated zsig37 product, wherein the lysine E-amino groups are PEGylated via an acyl linking group. An example of a connecting linkage is an amide. Typically, the resulting zsig37 peptide, polypeptide, or fusion protein will be at least 95% mono-, di-, or tri-pegylated, although some species with higher degrees of PEGylation may be formed depending upon the reaction conditions. PEGylated species can be separated from unconjugated species using standard purification methods, such as dialysis, ultrafiltration, ion exchange chromatography, affinity chromatography, and the like.

PEGylation by alkylation generally involves reacting a terminal aldehyde derivative of PEG with a zsig37 peptide, polypeptide, or fusion protein in the presence of a reducing agent. PEG groups can be attached to the polypeptide via a —CH₂—NH group.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the E-amino groups of the lysine residues and the a-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups. The present invention provides a substantially homogenous preparation of zsig37 monopolymer conjugates.

Reductive alkylation to produce a substantially homogenous population of monopolymer zsig37 peptide, polypeptide, or fusion protein conjugate molecule can comprise the steps of: (a) reacting a zsig37 peptide, polypeptide, or fusion protein with a reactive PEG under reductive alkylation conditions at a pH suitable to permit selective modification of the a-amino group at the amino terminus of the zsig37 peptide, polypeptide, or fusion protein, and (b) obtaining the reaction product(s). The reducing agent used for reductive alkylation should be stable in aqueous solution and able to reduce only the Schiff base formed in the initial process of reductive alkylation. Illustrative reducing agents include sodium borohydride, sodium cyanoborohydride, dimethylamine borane, trimethylamine borane, and pyridine borane.

For a substantially homogenous population of monopolymer zsig37 conjugates, the reductive alkylation reaction conditions are those which permit the selective attachment of the water soluble polymer moiety to the N-terminus of a zsig37 peptide, polypeptide, or fusion protein. Such reaction conditions generally provide for pKa differences between the lysine amino groups and the a-amino group at the N-terminus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer:zsig37 moiety need not be as large because more reactive groups are available. Typically, the pH will fall within the range of 3 to 9, or 3 to 6.

General methods for producing conjugates comprising a polypeptide and water-soluble polymer moieties are known in the art. See, for example, Karasiewicz et al., U.S. Pat. No. 5,382,657, Greenwald et al., U.S. Pat. No. 5,738,846, Nieforth et al., Clin. Pharmacol. Ther. 59:636 (1996), Monkarsh et al., Anal. Biochem. 247:434 (1997)).

The present invention contemplates compositions comprising a peptide, polypeptide, or fusion protein described herein. Such compositions can further comprise a carrier. The carrier can be a conventional organic or inorganic carrier. Examples of carriers include water, buffer solution, alcohol, propylene glycol, macrogol, sesame oil, corn oil, and the like.

7. Isolation of Zsig37 Peptides, Polypeptides, and Fusion Proteins

The peptides, polypeptides, and fusion proteins of the present invention can be purified to at least about 80% purity, to at least about 90% purity, to at least about 95% purity, or greater than 95% purity with respect to contaminating macromolecules, particularly other proteins and nucleic acids, and free of infectious and pyrogenic agents. The peptides, polypeptides, and fusion proteins of the present invention may also be purified to a pharmaceutically pure state, which is greater than 99.9% pure. In certain preparations, purified zsig37 molecules are substantially free of other polypeptides, particularly other polypeptides of animal origin.

Fractionation and/or conventional purification methods can be used to obtain preparations of synthetic zsig37 peptides, polypeptides, fusion proteins, and recombinant amino acid sequences purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are suitable. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method for polypeptide isolation and purification is a matter of routine design and is determined in part by the properties of the chosen support. See, for example, Affinity Chromatography: Principles & Methods (Pharmacia LKB Biotechnology 1988), and Doonan, Protein Purification Protocols (The Humana Press 1996).

The peptides, polypeptides, and fusion proteins of the present invention can also be isolated by exploitation of particular properties. For example, immobilized metal ion adsorption chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography, Protein A chromatography, and ion exchange chromatography (M. Deutscher, (ed.), Meth. Enzymol. 182:529 (1990)).

Additional variations in isolation and purification can be devised by those of skill in the art. For example, Sheppard, U.S. Pat. No. 6,265,544 (2001), and Sheppard et al., PCT publication No. WO00/48625 (2000), describe the isolation of Zsig37 polypeptides with N-terminal or C-terminal Glu-Glu (EE) tags using anti-EE Sepharose.

Zsig37 peptides, polypeptides, and fusion proteins may also be prepared through chemical synthesis, as described above. Zsig37 peptides, polypeptides, and fusion proteins may be monomers or multimers; glycosylated or non-glycosylated; PEGylated or non-PEGylated; and may or may not include an initial methionine amino acid residue.

8. Therapeutic Uses of Zsig37 Peptides, Polypeptides, and Fusion Proteins

Zsig37 peptides, polypeptides, and fusion proteins can be used to promote blood flow within the vasculature of a mammal. The administration of these molecules can reduce the number of platelets that adhere and are activated and the size of platelet aggregates. These molecules can be administered to any subject in need of treatment, and the present invention contemplates both veterinary and human therapeutic uses. Illustrative subjects include mammalian subjects, such as farm animals, domestic animals, and human patients. Zsig37 peptides, polypeptides, and fusion proteins can be administered prior to, during, or following an acute vascular injury in the mammal.

In one approach, the vascular injury is due to vascular reconstruction, including but not limited to, angioplasty, endarterectomy, coronary artery bypass graft, microvascular repair or anastomosis of a vascular graft. As an illustration, Zsig37 peptides, polypeptides, and fusion proteins can be administered prior to, during, or following endarterectomy (e.g., carotid endarterectomy). Also contemplated are vascular injuries due to trauma, stroke or aneurysm. In other methods, the vascular injury is due to plaque rupture, degradation of the vasculature, complications associated with diabetes and atherosclerosis. Plaque rupture in the coronary artery induces heart attack and in the cerebral artery induces stroke. Zsig37 peptides, polypeptides, and fusion proteins would also be useful for ameliorating whole system diseases of the vasculature associated with the immune system, such as disseminated intravascular coagulation (DIC) and SIDs. Additionally the complement inhibiting activity would be useful for treating non-vasculature immune diseases such as arteriolosclerosis.

A correlation has been found between the presence of C1q in localized ischemic myocardium and the accumulation of leukocytes following coronary occlusion and reperfusion. Release of cellular components following tissue damage triggers complement activation, which results in toxic oxygen products that may be the primary cause of myocardial damage (Rossen et al., Circ. Res. 62:572 (1998), and Tenner, Behring Inst. Mitt. 93:241 (1993)). Blocking the complement pathway was found to protect ischemic myocardium from reperfusion injury (Buerke et al., J. Pharm. Exp. Therp. 286:429 (1998)). The complement inhibition and C1q binding activity of zsig37 peptides, polypeptides, and fusion proteins would be useful for such purposes.

The collagen and C1q binding capabilities of zsig37 can be used to pacify damaged collagenous tissues preventing platelet adhesion, activation or aggregation, and the activation of inflammatory processes which lead to the release of toxic oxygen products. Without being limited to a particular theory, zsig37 may inhibit platelet adhesion, activation and/or aggregation by binding collagen related peptide (CRP), which has been demonstrated to selectively activate the platelet collagen receptor VI (GPVI) (Barnes et al., Curr. Opin. Hematol., 5(5):314-320 (1998)), and thus preventing GPVI from binding CRP (See Example 15). It is well known in the art that GPVI plays plays an important role in collagen-induced activation and aggregation of platelets, and people who are deficient in GPVI suffer from bleeding disorders (Jandrot-Perrus et al., Blood, 96(5):1798-1807 (Sept. 2000)). It is also well known in the art that platelet activation by collagen involves the highly-specific recognition of the Glycine-Proline-Hydroxyproline sequence by GPVI (Knight et al., Cardiovascular Research, 41(2):450-457 (Feb. 1999)). By rendering the exposed tissue inert towards such processes as complement activity, thrombotic activity and immune activation, zsig37 peptides, polypeptides, and fusion proteins would be useful to reduce the injurious effects of ischemia and reperfusion. Such injuries include, for example, trauma injury ischemia, intestinal strangulation, and injury associated with pre- and post-establishment of blood flow. Zsig37 peptides, polypeptides, and fusion proteins are also useful in the treatment of cardiopulmonary bypass ischemia and resuscitation, myocardial infarction and post trauma vasospasm, such as stroke or percutanious transluminal angioplasty, as well as accidental or surgical-induced vascular trauma. For example, zsig37 peptides, polypeptides, and fusion proteins can be used to treat acute coronary syndrome.

Zsig37 peptides, polypeptides, and fusion proteins are also useful to pacify prosthetic biomaterials and surgical equipment to render the surface of the materials inert towards complement activation, thrombotic activity or immune activation. Such materials include, but are not limited to, collagen or collagen fragment-coated biomaterials, gelatin-coated biomaterials, fibrin-coated biomaterials, fibronectin-coated biomaterials, heparin-coated biomaterials, collagen and gel-coated stents, arterial grafts, synthetic heart valves, artificial organs or any prosthetic application exposed to blood that will bind zsig37. Coating such materials can be performed using methods known in the art (see for example, Rubens, U.S. Pat. No. 5,272,074). The present invention also includes the use of zsig37 peptides, polypeptides, and fusion proteins to coat prosthetic biomaterials and surgical equipment, which have not been pre-coated with collagen, fibrin, gelatin, and the like.

Complement and C1q play a role in inflammation. The complement activation is initiated by binding of C1q to immunoglobulins (Johnston, Pediatr. Infect. Dis. J. 12:933 (1993); Ward and Ghetie, Therap. Immunol. 2:77 (1995)). Inhibitors of C1q and complement would be useful as anti-inflammatory agents. Such application can be made to prevent infection. Additionally, such inhibitors can be administrated to an individual suffering from inflammation mediated by complement activation and binding of immune complexes to C1q. Zsig37 peptides, polypeptides, and fusion proteins can be used to mediate wound repair, and enhance progression in wound healing by overcoming impaired wound healing. Progression in wound healing would include, for example, such elements as a reduction in inflammation, fibroblasts recruitment, wound retraction and reduction in infection.

The ability of tumor cells to bind to collagen may contribute to the metastasis of tumors. Inhibitors of collagen binding, such as Zsig37 peptides, polypeptides, and fusion proteins are also useful for mediating the adhesive interactions and metastatic spread of tumors.

Furthermore, zsig37 peptides, polypeptides, and fusion proteins can be therapeutically useful for anti-microbial applications. For example, complement component C1q plays a role in host defense against infectious agents, such as bacteria and viruses. C1q is known to exhibit several specialized functions. C1q also triggers the complement cascade via interaction with bound antibody or C-reactive protein (CRP). In addition, C1q interacts directly with certain bacteria, RNA viruses, mycoplasma, uric acid crystals, the lipid A component of bacterial endotoxin and membranes of certain intracellular organelles. C1q binding to the C1q receptor is believed to promote phagocytosis. C1q also appears to enhance the antibody formation aspect of the host defense system. See, for example, Johnston, Pediatr. Infect. Dis. J. 12(11):933 (1993). Thus, soluble C1q-like molecules may be useful as anti-microbial agents, promoting lysis or phagocytosis of infectious agents. Moreover, inhibition of inflammatory processes by polypeptides and antibodies of the present invention would also be useful in preventing infection at the wound site. Finally, zsig37 peptides, polypeptides, or fusion proteins can inhibit vegetative bacterial infection by reducing or preventing adhesion of bacteria to extracellular matrix proteins, such as collagen. As an example, Staphylococcus aureus has a collagen receptor that plays a role in endocarditis and septic arthritis.

Generally, the dosage of administered zsig37 peptide, polypeptide, or fusion protein will vary depending upon such factors as the subject's age, weight, height, sex, general medical condition and previous medical history. Typically, it is desirable to provide the recipient with a dosage of zsig37 peptide, polypeptide, or fusion protein, which is in the range of from about 1 pg/kg to 100 mg/kg, or 0.01 to 100 mg/kg (amount of agent/body weight of subject), although a lower or higher dosage also may be administered as circumstances dictate. In applications such as balloon catheters, a typical dose range would be 0.05-5 mg/kg of subject. Doses for specific compounds may be determined from in vitro or ex vivo studies in combination with studies on experimental animals. Concentrations of compounds found to be effective in vitro or ex vivo provide guidance for animal studies, wherein doses are calculated to provide similar concentrations at the site of action.

For pharmaceutical use, the zsig37 peptides, polypeptides, and fusion proteins of the present invention can be formulated with pharmaceutically acceptable carriers for administration via intravenous, intraarterial, intraperitoneal, intramuscular, subcutaneous, intrapleural, or intrathecal routes, by perfusion through a regional catheter, or by direct intralesional injection. When administering therapeutic proteins by injection, the administration may be by continuous infusion or by single or multiple boluses.

Additional routes of administration include oral, topical, inhalant, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Ilium, Adv. Drug Deliv. Rev. 35:199 (1999)). Dry or liquid particles comprising a zsig37 peptide, polypeptide, or fusion protein can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer a zsig37 peptide, polypeptide, or fusion protein (Potts et al., Pharm. Biotechnol. 10:213 (1997)).

Preferably, administration is made at or near the site of vascular injury. In general, pharmaceutical formulations will include a zsig37 peptide, polypeptide, or fusion protein in combination with a pharmaceutically acceptable carrier, such as saline, buffered saline, 5% dextrose in water, and the like. A pharmaceutical composition comprising a zsig37 peptide, polypeptide, or fusion protein can be formulated according to known methods to prepare pharmaceutically useful compositions, whereby the therapeutic proteins are combined in a mixture with a pharmaceutically acceptable carrier.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995). Formulations may further include one or more excipients, preservatives, solubilizers, buffering agents, albumin to prevent protein loss on vial surfaces, etc. Methods of formulation are well known in the art and are disclosed, for example, in Gennaro (ed.), Remington's Pharmaceutical Sciences, 19th Edition (Mack Publishing Company 1995).

As used herein a “pharmaceutically effective amount” of a zsig37 peptide, polypeptide, or fusion protein is an amount sufficient to induce a desired biological result. The result can be alleviation of the signs, symptoms, or causes of a disease, or any other desired alteration of a biological system. For example, an effective amount of a zsig37 polypeptide is that which provides either subjective relief of symptoms or an objectively identifiable improvement as noted by the clinician or other qualified observer. Such an effective amount of a zsig37 polypeptide would provide, for example, inhibition of collagen-activated platelet activation, or the complement pathway, including C1q, increased localized blood flow within the vasculature of a patient, or reduction in injurious effects of ischemia and reperfusion.

A pharmaceutical composition comprising a zsig37 peptide, polypeptide, or fusion protein can be furnished in liquid form, in an aerosol, or in solid form. Liquid forms, are illustrated by injectable solutions and oral suspensions. Exemplary solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Liposomes provide another means to deliver therapeutic zsig37 peptides, polypeptides, or fusion proteins to a subject intravenously, intraperitoneally, intrathecally, intramuscularly, subcutaneously, or via oral administration, inhalation, or intranasal administration.

The present invention also contemplates chemically modified zsig37 peptides, polypeptides, or fusion proteins in which the zsig37 amino acid sequence is linked with a polymer, as discussed above.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

A subject can be treated with a pharmaceutical composition comprising a zsig37 peptide, polypeptide, or fusion protein that is in the form of an oligomer. Illustrative oligomers include trimers, hexamers, 9mers, and 18mers. Pharmaceutical compositions can also comprise a mixture of zsig37 oligomers. For example, a pharmaceutical composition can comprises a mixture of trimers and hexamers of a polypeptide that comprises amino acid residues 26 to 281 of SEQ ID NO:2. In particular trimer-hexamer mixtures, the ratio of trimer/hexamer may be in the range of about 1/99, 2/98, 3/97, 4/95, 5/95, 6/94, 7/93, 8/92, 9/91, 10/90, 11/89, 12/88, 13/87, 14/86, 15/85, 16/84, 17/83, 18/82, 19/81, 20/80, 25/75, 30/70, 40/60, 50/50, 60/40, 70/30, 75/25, 80/20, 81/19, 82/18, 83/17, 84/16, 85/15, 86/14, 87/13, 88/12, 89/11, 90/10, 91/9, 92/8, 93/7, 94/6, 95/5, 96/4, 97/3, 98/2, or 99/1. Certain pharmaceutical compositions comprise a mixture of oligomers in which the trimer/hexamer ratio lies in the range of about 5/95 to about 20/80.

A zsig37 peptide, polypeptide, or fusion protein can be administered to a subject with or without an additional therapeutic agent. Suitable therapeutic agents for use in combination with a zsig37 peptide, polypeptide, or fusion protein include (1) agents that affect platelet function (e.g., aspirin 7 cox II inhibitors, Clopidigrel, ticlopidine, GPIIbIIa inhibitors, GPIb inhibitors, anti-von Willebrand factor drugs, and the like), (2) agents that inhibit or promote blood coagulation factors such as Factors IIa, V(a), VII(a), VII(a), IX(a), X(a), XI(a), XII(a), and XIII(a), (3) blood coagulation factor inhibitors (e.g., heparins (fractionated and un-fractionated), dicoumarin, warfarin, anti-thrombin III, heparin cofactor, tissue factor pathway inhibitor, FVIIai, nematode anticoagulant protein C2, tick anti-coagulant, Protein C, Protein S, pentasaccharide, DX-9065a, sodium N-(8[2-hydroxybenzoyl]amino)caprylate/heparin, hirudin, bivalirudin, argatroban, H376/95 (a pro-drug formulation of melagatran), and the like, as well as thrombomodulin and thrombomodulin mutants, truncations, chimeras, and the like, and (4) agents that promote or accelerate fibrinolysis (e.g., tissue plasminogen activators, streptokinase, straphlokinase, (pro-)urokinase, Protein C, Protein S, thrombomodulin and thrombomodulin mutants, truncations, chimeras, and the like). These therapeutic agents can be administered before, concomitant with, or after the administration of a zsig37 peptide, polypeptide, or fusion protein.

Combination therapy can be used to treat disorders and diseases described herein. For example, the combination of a zsig37 peptide, polypeptide, or fusion protein with at least one other therapeutic agent can be used to treat acute myocardial infarction.

Pharmaceutical compositions that include a zsig37 therapeutic agent may be supplied as a kit comprising a container that comprises a zsig37 peptide, polypeptide, or fusion protein. Therapeutic polypeptides can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical composition. Moreover, such information may include a statement that the zsig37 peptide, polypeptide, or fusion protein composition is contraindicated in patients with known hypersensitivity to either the zsig37 moiety or the immunoglobulin moiety.

The present invention, thus generally described, will be understood more readily by reference to the following examples, which are provided by way of illustration and is not intended to be limiting of the present invention.

### EXAMPLE 1

### Adhesion and Proliferation Assays

The ability of zsig37 to stimulate adhesion and spreading of TF-1 cells was assayed as follows. A series of dilutions were prepared from C-terminal Glu-Glu-tagged zsig37, from 10 to 0.0625 ’g/ml, in either PBS or ELISA coating buffer (0.1 M NaCO₃) and each was plated into a 96 well plate (Costar; Pleasanton, Calif.) at 100 μl/well. The plates were incubated at 37° C., 5% CO₂ for 2 hours. The plates were then washed 3× with RPMI/10% FBS (RPMI 1640, 2 mM L-glutamine, 110 μg/ml sodium pyruvate, PSN and 10% heat inactivated fetal bovine serum) and allowed to block for 15 minutes.

TF-1 cells (derived from acute myeloid leukemia cells) were resuspended in RPMI/10% FBS and plated into at 10,000 cells/well into the zsig37CEE-coated 96 well plates at a final volume of 120 μl/well. The plate was incubated at 37° C. under 5% CO₂ for 2 hours. The plates were then washed 3× with PBS and 200 μl/well growth media (RPMI/10% FBS, 5 ng/ml GM-CSF) was added. The cells were microscopically inspected before and after the wash.

A dye incorporation assay was also used to measure the number of adherent cells based on a colorimetric change and an increase in fluorescent signal. ALAMAR BLUE (AccuMed; Chicago, Ill.) was added to the 96 well plates and the cells were incubated at 37° C. under 5% CO₂ overnight. The plates were then scanned using a fluorometer with excitation wavelength of 544 nm and emission wavelength of 590 nm. There were more adherent cells on the C-terminal Glu-Glu tagged (zsig37CEE)-PBS coated plates than on the zsig37CEE-0.1 M NaCO₃ coated plates. Addition of soluble zsig37 did not block adhesion of cells to the bound zsig37.

A second assay was performed with TF-1, DA-1, an IL-3 dependent cell line derived from the lymph node of a mouse with a B-cell lymphoma by outgrowth in IL-3 media, pre-B (p53−/− mouse marrow cells, IL-7 dependent, B220+, Thy1 low, Sca-1+), and A7BaF-3 cell lines as described above at 5,000 cells/well. BHK cells were also plated at 500 cells/well. Zsig37 enhanced the growth of A7-BaF-3 cells and slightly inhibited growth of DA-1 cells.

### EXAMPLE 2

### Cell-Based Assays

Zsig37 polypeptides were assayed in a high throughput, in vitro assay to identify substances that selectively activate cellular responses in immortalized osteoblast cell lines. A mature osteoblast cell line derived from p53−/− (deficient) mice, CCC4, that is transfected with a plasmid containing an inducible serum response element (SRE) driving the expression of luciferase was used in the assay. These cells also express endogenous PTH, PDGF and bFGF receptors. The stimulation of the SRE and thus the expression of luciferase in the CCC4 cells indicates that the chemical entity is likely to stimulate mitogenesis in osteoblasts.

CCC4 lines were trypsinized and adjusted to 5×10⁴ cells/ml in plating medium (alpha-MEM, 1% heat inactivated fetal bovine serum, 1 mM Na pyruvate and 2 mM L-glutamate) and plated (200 μl/well) into Dynatech Microlite opaque white microtiter plates (Dynatech, Chantilly, Va.) and incubated overnight at 37° C., 5% CO₂. The growth medium was then aspirated and replaced with 50 μl/well assay medium (F-12 HAM, 0.5% bovine serum albumin, 20 mM HEPES, 1 mM sodium pyruvate and 2 mM L-glutamate). Serial dilutions of zsig37 were made in assay medium (0.29-1000 ng/ml final assay concentration) and added to the wells. Zsig37 samples were assayed in triplicate. Serum (negative) and bFGF (positive) controls were also used. The final concentration of bFGF was 3 ng/ml. Controls were assayed in quadruplicates. The plates were incubated for four hours at 37° C., 5% CO₂. The assay medium was then aspirated and the plates were rinsed once with PBS. To each well was then added 25 μl of lysis buffer (Luciferase Assay Reagent, E1501, Promega Corp.; Madison, Wis.). The plates were incubated for 15 minutes at room temperature. Fifty microliters/well of luciferase substrate (Luciferase Assay Reagent, E1501, Promega Corp.) was added and the Luciferase activity was detected using a Labsystems LUMINOSKAN at 2 second/well following a one second delay. The average basal (uninduced) signal was subtracted from readings as a percentage of the maximal induction produced by 3 ng/ml bFGF. These studies showed that zsig37 stimulates the expression of luciferase in this assay indicating that zsig37 stimulated osteoblasts. Zsig37 stimulates at 73 to 75% maximal at 1000 ng/mI.

### EXAMPLE 3

### Vasodilatation of Aortic Rings

The effect of zsig37 on vasodilatation of aortic rings was measured according to the procedures of (Dainty et al., J. Pharmacol. 100:767 (1990), and Rhee et al., Neurotox. 16:179 (1995)). Briefly, aortic rings 4 mm in length were taken from 4 month old Sprague Dawley rats and placed in modified Krebs solution (118.5 mM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). The rings were then attached to an isometric force transducer (Radnoti Inc.; Monrovia, Calif.) and the data recorded with a Ponemah physiology platform (Gould Instrument systems, Inc.; Valley View, Ohio) and placed in a 10 ml tissue bath oxygenated (95% O₂, 5% CO₂) modified Krebs solution. The tissues were adjusted to one gram resting tension and allowed to stabilize for one hour before testing.

The rings were tested by 5 μl additions of 1×10⁻⁷ M norepinepherin (Sigma Chemical Co.; St. Louis, Mo.) to a final concentration of about 1×10⁻⁹ M and Carbachol, a muscarinic acetylcholine agonist (Sigma Chemical Co.) at 2×10⁻⁷ M final, to test the integrity of the rings. After each test, the rings were washed three times with fresh buffer, five minutes between washes and allowed to rest one hour. To test for vasodilatation, the rings were contracted to two grams and allowed to stabilize for fifteen minutes. Zsig37 was then added to one, two, or three of the four baths, without flushing, and tension on the rings was recorded and compared to the control rings. The rings were then tested for contraction with norepinepherin as described above. Rings were tested at 323, 162, and 81 ng/ml zsig37 but a dose response could not be determined. In order to evaluate the statistical significance of the data, a contingency test was performed on all the zsig37 and control rings using dilation as a determinant. Of 10 of the 12 rings tested with zsig37 vasodialated as did two of the seven controls. The Fisher exact P value is 0.045. It was concluded that zsig37 induces vasodilatation in norepinepherin contracted aortic rings.

### EXAMPLE 4

### Binding of zsig37 to Matrix Proteins

An ELISA (Enzyme-linked Immunosorbant Assay) was used to measure binding of zsig37 to complement C1q, and to the following matrix proteins: Bovine Collagen Type I (Becton Dickinson; Lincoln Park, N.J.), laminin, vitronectin, fibronectin, human collagen Types II, III, IV, V, VI (Chemicon International; Temecula, Calif.). BSA V (Sigma Chemical Co.) was used as a negative control. Just prior to use, the proteins were diluted in 2× PBS (Phosphate Buffered Saline, Sigma Chemical Co.) to 100 μg/ml and adjusted to pH 7.2 with 0.1 N NaOH. Each protein sample was plated in quadruplicate (100 μl/well) into a 96 well plate. The plate was allowed to dry overnight in a laminar flow hood and washed three times with 400 μl of 5 mg/ml BSA in 1× PBS and blotted dry. Zsig37 was FITC labeled according to manufacturer's instruction (Pierce; Rockford, Ill.). Into each well was added 100 μl of 1.8 μg/ml zsig37-FITC in 5% BSA, PBS. The plates were incubated for 1.5 hours at room temperature then washed 3 time with 5% BSA, PBS. To each well was then added 100 μl of 1:400 mouse anti-FITC/Biotin (Sigma Chemical Co.). The plate was incubated 1.5 hours at room temperature and washed three times with 5% BSA, PBS. The plate was then incubated with 100 μl of 1:1000 streptavidin/HRP (Amersham; Piscataway, N.J.) for one hour and washed three times with 5% BSA, PBS. The plate was then developed using SUPERSIGNAL Ultra (Pierce; Rockford, Ill.) according to manufacturer's instruction. After reacting for one minute, surplus liquid was removed from the plate by inverting the plate and patting dry. The plate was exposed to X-ray film (Kodak; Rochester, N.Y.).

The results of this screen indicate that only fibronectin and the collagens I, II, III, IV, V and VI bind significantly to zsig37-FITC. Such binding was not seen with laminin, vitronectin, or the BSA control.

### EXAMPLE 5

### Specificity of Zsig37 Binding to Collagen Type VI and to Complement C1q

The ELISA assay for binding, described above, was modified to quantitatively evaluate binding. Zsig37-FITC, in a range of 0.4 to 4 μg/ml, was bound to 10 μg of collagen type VI (Chemicon International) as described above. The luminescence from the SUPERSIGNAL reagent was read on a Wallac 1420 plate reader (Wallac; Gaithersburg Md.) and the intensity used as a quantitative measure of the zsig37-FITC bound to the ELISA plate.

The results showed that the binding of zsig37 to collagen type VI fits a typical hyperbolic binding curve, and that bound Zsig37-FITC plated at 0.4 μg/ml can be competed off the collagen by the addition of unlabeled Zsig37 in a range of 0.8 to 8 μg/ml. These data indicate that binding is specific for domains on collagen type VI and is concentration dependent.

Zsig37-FITC at 0.2 μg/ml was shown to bind to complement C1q (Sigma Chemical Co.) at 0.1 to 10 μg/ml by the method described above. The amount of binding was concentration dependent and saturable.

### EXAMPLE 6

### Complement Inhibition by Zsig37

Complement assays were performed in 96 well round bottom plates. Gelatin Veronal buffer containing magnesium and calcium (141 mM NaCl, 1.8 mM sodium barbitol, 3.1 mM barbituric acid, 0.1% bovine gelatin, 0.5 mM MgCl₂ and 0.15 mM CaCl₂) was used for all serum and inhibitor dilutions as well as erythrocyte suspensions. Fifty microliters of standardized human Complement serum (Sigma Chemical Co.), diluted 1/37.5 (for a final dilution of 1/150) was added to each well. The inhibitor was added in triplicate, 50 μl/well. The serum and inhibitor were incubated for thirty minutes at room temperature. The assay was initiated by the addition of 100 μl of 2×10⁸/ml unsensitized sheep erythrocytes (Colorado Serum Co.; Denver, Colo.), sensitized sheep erythrocytes, sensitized using the Hemolysin manufacturer's protocol (BioWhittaker Inc.; Walkersville, Md.) and rabbit erythrocytes containing 16 mM EGTA, and 4 mM Mg⁺⁺. A human serum dilution series from 1/50 to 1/400 was also plated as an activity control. Erythrocytes, lysed with distilled water and diluted to 100, 75, 50, 25, and 12.5 percent lysis, were used to quantify complement percent lysis. The plate was sealed and incubated at 37° C. for one hour with mixing every 15 minutes. The reaction was stopped by the addition of 220 mM EDTA, 20 μl/well and the plates centrifuged at 1500×G for 10 minutes. One hundred microliters of supernatant was removed from each well and transferred to a 96 well flat bottom plate for analysis. The plate was read at 415 nM and percent lysis was calculated.

Zsig37 was effective in inhibiting the classical pathway with both sensitized and unsensitized sheep erythrocytes. There was no apparent inhibition of the alternate pathway tested with rabbit erythrocytes and EGTA. The mechanism of inhibition is undetermined but because C1q binds zsig37, C1 is the most likely target.

### EXAMPLE 7

### Inhibition by Zsig37 of Platelet Collagen Activation

Blood was drawn from healthy volunteers into tubes containing sodium citrate, maintained at room temperature, and used within four hours of drawing. Whole blood was analyzed for platelet activation using a Chrono-Log 560A Whole Blood Lumi-Aggregometer (Chrono-Log Corp.; Haverton, Pa.) according to manufacturer's instructions. For each test point, 500 μl of blood were added to a reaction tube containing a stir bar and 500 μl of isotonic saline containing zsig37 at concentrations from 0 to 20 μg/ml. The mixture was incubated for four minutes followed by platelet activation initiated by the addition of 5 μl of 1 mg/ml cross-linked collagen (Chrono-Log Corp.) to the blood/zsig37 mixture. Inhibition of activation by ADP (final concentration 10 μM), and thrombin (final concentration lU/ml) were tested in a similar way.

Inhibition of collagen-mediated platelet activation by zsig37 showed a dose dependent relationship between 5 and 20 μg/ml. The inhibition was selective for collagen activation and had no effect on activation stimulated by ADP or thrombin.

### EXAMPLE 8

### Activity of Zsig37 in Carotid Artery Injury Model With Rabbits and Non-Human Primates

Zsig37 was administered in a modified rabbit carotid artery injury model (Folts et al., Circulation 79:116 (1989), and Golino et al., Thrombosis and Haemostasis 67:302 (1992)) to determine the degree of protection offered in preventing vascular occlusion following a crush injury.

Thirty-four male New Zealand White rabbits, approximately three to six months old (R&R Rabbitry; Stanwood, Wash.) were divided into two groups. Fifteen rabbits received doses of zsig37 ranging from 2-13.5 μg/kg and 19 control rabbits were injected with PBS or equivalent amounts of PBS or zsig39, another adipocyte complement related protein (WO99/10492). The rabbits were anesthetized with ketamine (50 mg/kg, IM) and maintained on halothane inhalation anesthesia for the duration of the study. The hair was shaved from the ears and neck and an angiocatheter was placed in the marginal ear vein for IV support. A midline incision was made in the neck and the carotid artery was accessed. Approximately 5 cm of the common carotid artery proximal to the internal/external bifurcation was exposed via blunt dissection away from the surrounding tissue and any visible side branches were cauterized. A flow probe (Transonic Systems, Inc.; Ithaca N.Y.) was placed distal to the anticipated injury site and a baseline blood flow was established. A 2.5-3.0 cm section of the vessel was then isolated from circulation using atraumatic vascular clamps. Following removal of the blood from the vessel segment, 0.4 ml of zsig37 in 0.9% sodium chloride or 0.04 ml 0.9% sodium chloride as a control, was injected into the empty vessel segment using a 30 G needle. The vessel was left undisturbed for a five-minute pre-injury treatment. A 1.0 cm crush injury was then inflicted into the center of the vessel segment using a guarded hemostat and left undisturbed for 10 minutes. The vessel clamps were then removed and blood flow reestablished. Blood flow was monitored continuously for 60 minutes after which time the rabbits were euthanized and the vessel excised for histological analysis.

No dose dependency was seen at these concentrations. A meta analysis of all zsig37 doses resulted in significant increase in time patent when compared to controls in an unpaired t-test (P=0.019).

The mean percent time patent for the combined groups of negative control animals, as determined from blood flow tracings, was 13.5% with a standard error of ±1.7%. The mean percent time patent for the combined zsig37 treated groups of animals, as determined from blood flow tracings, was 37.2% with a standard error of ±10.3%.

In a second series of experiments, fluoresceinated zsig37 was used in the injured carotid artery model. Male New Zealand White rabbits were anesthetized as above. Via an incision in the neck, the carotid artery was exposed and approximately 5 cm of the vessel isolated from the surrounding consecutive tissue. Blood was evacuated from the isolated segment and atraumatic vascular clips were applied. Approximately 0.05 ml of fluoreceinated zsig37 (concentration 100 μg/ml) was injected into the isolated segment to completely fill the vessel using a 30 g needle. After an exposure period of five minutes, the vessel was injured and the exposure continued for another 110 minutes before the clips were removed and blood flow reestablished. The animals were euthanized as described above at 1, 10, and 60 minutes post-reestablishment of blood flow and the vessels collected and formalin fixed for histological evaluation.

Labeled zsig37 preferentially bound to molecules in the media of the injured vessels. Labeled zsig37 did not bind to areas of the vessel that were uninjured. Since no difference was observed in the amount of labeled zsig37 bound to the tissues in the 1 minute vs. the 60 minute collection time point, the time of blood flow prior to vessel collection does not appear to affect the amount of zsig37 that remains bound to the tissue. This may indicate that zsig37 tightly binds to the injured vessel and is not washed off by the reestablished blood flow.

The effect of zsig37 on blood flow dynamics following vascular injury in a rabbit iliac artery crush injury/stenosis model was also evaluated. Young adult male New Zealand White rabbits were anesthetized as described above. Via an abdominal incision, the aorto-iliac bifurcation was exposed and each iliac freed of surrounding tissues and the main branches ligated. Each iliac was fitted with an ultrasound flow probe to monitor blood flow through the vessel. Based on blood flow data, one iliac was selected to be used for the injury and the other was catheterized for delivery of the test sample. Rabbits were divided into dose groups of 6 animals/group. Test sample doses containing zsig37 increased in half-log increments from 3-1000 μg/kg over the selected infusion period. The test samples infusion was initiated followed by creation of a critical stenosis that reduced blood flow through the vessel by approximately 50%. After creation of the stenosis and a period of blood flow stabilization, the vessel was injured by crushing the vessel between the jaws of a smooth needle holder. The infusion was continued post-injury for a set period of time, 10-20 minutes. Blood flow through the injured vessel was monitored for 60 minutes post-injury. The animals were euthanized at he conclusion of the study period. The lower section of the abdominal aorta and each iliac were collected and formalin fixed for histological evaluation.

Blood flow parameters determined from the flow tracings, included mean flow post-stenosis, mean flow post-injury, and time the vessel remained patent. These data suggest that there is a tendency for zsig37 to promote increased patency time with increased dose up to 300 μg/kg over a 60 minute period.

In another study, rabbits received the vascular injury and were treated with either 1000, 350 or 250 μg/kg zsig37 hexamer. The animal receiving the highest dose had the most rapid increase in flow—starting from occlusion—and the animal receiving the lowest dose had the slowest increase in blood flow. These results demonstrate a dose response for the anti-thrombotic effect of zsig37 hexamer, and it further shows that zsig37 possesses thrombolytic activity.

The effect of zsig37 on platelet rich thrombus formation induced by vascular injury (Folts Model) was also tested with cynomolgus monkeys. In the absence of treatment, blood flow in the vessels decreased to zero flow, indicating an occlusion of the artery. In contrast, cynomolgus monkeys that received the vascular injury and were treated with 1.0 mg/kg zsig37 had vessels that were fully patent by 20 minutes following treatment and remained open. When cynomolgus monkeys received the vascular injury and were treated with 0.5 mg/kg zsig37, their vessels were fully patent by 30 minutes following treatment and then remained open.

### EXAMPLE 9

### Relaxation of Serotonin-Induced Rat Aortic Ring Contractions

Male, Sprague-Dawley rats, approximately 3 months of age, were lightly anesthetized with CO₂ and then decapitated. The thoracic aorta was then rapidly removed and placed in a modified Kreb's-Henseleit buffer (NaCl, 118.2 mM; KCl, 4.6 mM; CaCl₂, 2.5 mM; MgSO₄, 1.2 mM; NaHCO₃, 24.8 mM; KH₂PO₄, 1.2 mM; and glucose, 10.0 mM). From each rat, four 2-3 mm aortic ring sections were cut after discarding the rough end of the aorta. In some experiments the endothelium was denuded, prior to cutting ring sections, by rubbing the lumen of the aorta along a 21 gauge needle. Denudation of the endothelium was verified by the addition of the acetylcholine analogue, carbachol, prior to determining zsig37 concentration-dependent responses. In the absence of the endothelium, carbachol does not vasorelax constricted vascular ring sections.

The rings were fixed and connected to force displacement transducers in oxygenated (95% O₂, 5% CO₂), jacketed, glass organ baths kept at 30° C. in modified Kreb's-Henseleit buffer, pH 7.4. Resting tension was set at 1 gm, and continually re-adjusted to 1 gm over a one-hour incubation period. Fresh oxygenated modified Kreb's-Henseleit buffer was added to the baths every fifteen minutes during the resting incubation period. At the end of the one-hour incubation, the ring sections were contracted by the addition of 10 μM serotonin. After maximum contraction had been reached, approximately 15-20 minutes after the addition of the serotonin, cumulative concentration response curves for zsig37 were constructed. Zsig37 was added to 5 ml baths in volumes from 5 up to 150 μls, for final concentrations ranging from 1 ng/ml up to 40 μg/ml. Viability of the ring sections was verified at the end of the concentration response by the addition of forskolin (2.5 μM or 25 μM) or nitroglycerin (22 μM).

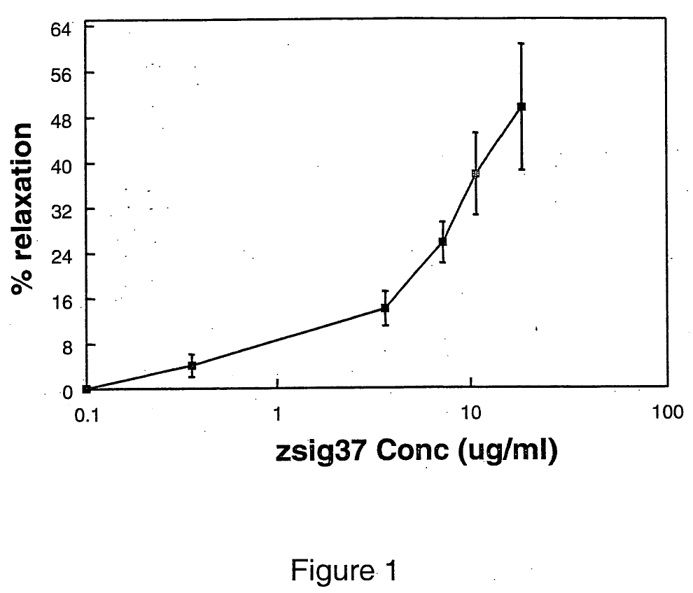

Addition of zsig37 induced a concentration-dependent vasorelaxation of serotonin-contracted rat aortic sections with and without an intact endothelium (FIG. 1). Relaxation in response of zsig37 was first observed at concentrations above 100 ng/ml. Relaxation was observed approximately 30-60 seconds after the additions of each zsig37 concentration to the bath, and relaxation responses plateaued within 3-5 minutes after the addition of zsig37. The character of the relaxation response to zsig37 indicates that the vasorelaxation is a receptor-second messenger mediated event. Additionally, the ability of zsig37 to vasorelax endothelium-denuded aortic sections indicates that zsig37 acts directly on the smooth muscle cells to elicit the vasorelaxant response.

### EXAMPLE 10

### Indium Labeled Zsig37

A ten-fold molar excess of DTPA (diethylenetriamine pentaacetic acid), a chelating agent, was reacted with zsig37. The resultant product was delivered into a 10,000 MWCO Slide-A-Lyzer dialysis cassette, equilibrated in a 0.1 M Hepes buffer, pH 7.0 for a minimum of 4 hours or overnight, with at least one buffer exchange. The zsig37/DTPA was removed from the cassette and reacted with ¹¹¹In at 150 gCi/mg at room temperature for 30 minutes with rocking. The zsig37¹¹¹In product was desalted to remove any unbound ¹¹¹In and Hepes buffer using a PD-10 column equilibrated with 0.1 M Acetate pH 6.0, or 120 mM NaCl. Five hundred microliter fractions were collected and monitored for radioactivity on a gamma counter. The fractions containing protein (radiation) were pooled and 500 mM Na Phosphate pH 7.4 was added to the pooled volume to a final concentration of 10 mM.

¹¹¹In-labeled zsig37 was administered at 30, 100, 300 and 1000 μg/kg in a modified rabbit carotid artery injury model as described above. ¹¹¹In-labeled zsig37 was detected in the highest concentrations at the site of the injury and in liver and kidney.

### EXAMPLE 11

### Expression of Zsig37 in Monocytes

The presence of zsig37 transcripts in monocytes was investigated by RT PCR. First strand cDNA was made from 1 μg total RNA using Superscript II reverse transcriptase (Life Technologies, Inc.) according to the manufacturer's instructions. Ten percent of the first strand cDNA was used as the template in a subsequent PCR reaction using zc22288 (5′ TCCCCTTTCA AGATAGTGAT GTTG 3′; SEQ ID NO:13) and zc22289 (5′ CATGAAAAAT ACAGGCCCAG TCA 3′; SEQ ID NO:14). Cycling conditions consisted of one cycle at 94° C. for 2 minutes, 45 cycles at 94° C. for 15 seconds, 60° C. for 30 seconds, and 68° C. for 45 seconds, followed by one cycle at 72° C. for 7 minutes. The reaction contained 200 nM dNTPs (Perkin Elmer), 400 nm each sense and antisense primers, 1× Rediload (Reasearch Genetics), 1× Advantage 2 cDNA polymerase mix buffer (Clontech), and 1× of Advantage 2 cDNA polymerase mix Zsig37 Expression was observed in activated monocytes.

### EXAMPLE 12

### The Effect of Zsig37 on Blood Flow in an Atherosclerotic Folts Model

Introduction. The Folts cyclic flow model (Circulation. June 1991;83(6 Suppl):IV3-14.) was adapted to study cyclic flow variations in an atherosclerotic rabbit femoral artery, as opposed to healthy vessels Folts, J D, Cardiovasc Res. April 1999;42(1):6-8; Maalej et al., J Thromb Thrombolysis. July 1998;5(3):231-238; and Woolf et al., “Interrelationship between atherosclerosis and thrombosis,” in: Fuster V, editor, Thrombosis in cardiovascular disorders, Saunders, N.Y., WB, 1992, pp. 50-55. Although the crush injury results in blood being exposed to the constituents of the vessel wall, there would be relatively less tissue factor released due to the absence of atherosclerotic plaque.

In the present experiment the standard Folts model was modified to study restenosis in rabbits. In this design, New Zealand White rabbits that normally are not prone to atherosclerosis were fed an atherogenic diet (e.g., 2% cholesterol and 6% coconut oil) for two weeks, underwent balloon denudation of an artery segment and then were continually fed the atherogenic diet. Within three weeks, atherosclerotic plaque had accumulated in the balloon-injured area. At this time, the animal was prepped for surgery and the atherosclerotic vessel was injured via a crush procedure and a Folts model study was performed.

Eleven male New Zealand White rabbits weighing 2.0-2.5 kg were used in this study. Atherosclerosis was developed in the right iliac and femoral arteries following a protocol described by Faxon et al., Am J. Cardiol., 1984, 53:72C-76C; and Faxon et al., Atherosclerosis. 1982, 2:125-133), with the following modifications. Animals were placed on an atherogenic diet consisting of standard rabbit chow supplemented with 2.0% cholesterol and 6% coconut oil (Research Diets, NJ) for two weeks prior to surgery. On the day of surgery, animals were anesthetized by using an intramuscular injection of ketamine (50 mg/kg) and prepared for sterile surgery. All animals underwent primary iliac and femoral artery deendothelialization using a 2F Fogarty Emobolectomy balloon catheter. Three weeks following balloon injury, animals were started on a modified Folts protocol. Blood samples were collected weekly to determine plasma cholesterol levels.

Protocol for Folts procedure. Animals were fasted overnight and then pre-anesthetized with ketamine hydrochloride and maintained on isoflurane inhalation anesthesia for the remainder of the study. A blood sample for CBC and coagulation assays was collected prior to the surgical procedure.

The following procedures were performed so that animals had a catheter for blood pressure measurement in the left carotid artery, an infusion catheter in the right jugular vein, and a flow probe on the right iliac artery. Animals were administered 100 U/kg heparin. Blood for APTT values was collected prior to and following heparin dosing. The left carotid artery was exposed and a catheter was inserted so that the tip was in the aorta. The jugular vein was exposed and a catheter was inserted. The right femoral and iliac arteries were exposed and all branches tied off. A flow probe was placed around the right iliac artery. Distal to this probe, a stenosis was placed on the artery to reduce the baseline blood flow by 10-15%. The stenosis was moved and a crush injury was made using a fine pair of hemostats and then the stenosis was repositioned over the injury. The flow rate was monitored and the vessel tapped to release the thrombus when the flow rate approached 0.7 ml/min. This monitoring and tapping of the vessel to restore flow was made until a baseline response was established. Once a predictable baseline response had been achieved, test or control articles were administered by bolus. Blood flow through the injured vessel was monitored for 60 minutes post bolus infusion. During the first 30 minutes of this period, the vessel was tapped as needed to release any occlusive thrombus (flow<0.7 mLamin). During the latter 30 minutes, the vessel was not tapped and any thrombus was allowed to accumulate. Blood pressure was monitored throughout and, prior to termination, blood samples for coagulation factors were collected. Using this protocol, 1 mg/kg zsig37 (n=8) was compared to vehicle (1 mg/kg BSA, n=3).

Histological Tissue Preparation. At the termination of the study, the injured vessel was flushed with saline and formalin, removed, kept in formalin. The vessel was embedded in paraffin, sectioned and stained with trichrome to highlight the collagen. Additional sections were cut, and using immunohistochemical techniques, stained for the presence of zsig37.

Statistical Analysis. All values are expressed as mean±SEM. A non-paired Student's t test was performed to detect differences between subgroups. A value of P≦0.05 was considered statistically significant.

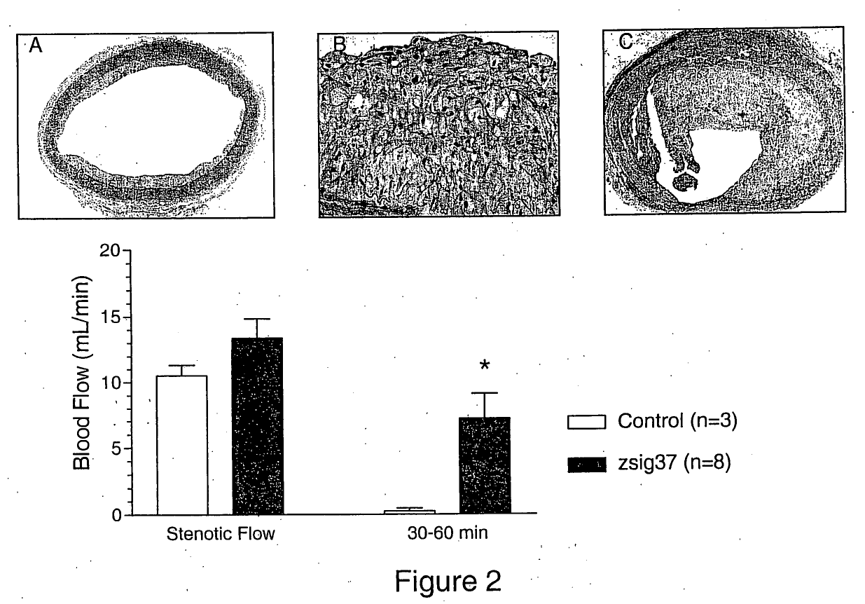

Results. The data analyzed for each animal were from the final 30 minutes. During this period, occlusive thrombi were allowed to form and flow varied from near zero for the albumin treated animals to continuously patent for the zsig37 treated animals (FIG. 2). The difference in flow between the groups was significant (p=0.0305; unpaired, one-tailed, t-test; t=2.140, df=9).

These data show that zsig37 can be effective in preventing platelet rich thrombi from forming in the presence of an atherosclerotic lesion. The model was developed to meet the need for closer simulation of plaque rupture as would be seen in myocardial infarction. The animals were placed on the atherogenic diet prior to surgery to ensure adaptation to the diet. This may have contributed to the entire segment of balloon-injured vessel being coated with atherosclerotic lesion by 3 weeks. These data show that zsig37 can be used to prevent platelet aggregation in animals with atherosclerotic lesions.

### EXAMPLE 13

### Use of a Template Bleed Procedure in Macaca fascicularis to Evaulate Effects of zsig37

Introduction. Template bleeding time is a laboratory test used in clinical medicine to measure primary hemostatic competency and the rate at which a platelet thrombus is formed. This test involves controlling the blood pressure in the test extremity and producing a standardized length and depth wound. The wound is carefully blotted with filter paper, taking care not to disturb the developing clot and the time to cessation of bleeding recorded. Bleeding time determined in this manner may be prolonged in thrombocytopenia, platelet dysfunction, vonWillebrand disease, hypofibrinongenemia and anticoagulant therapy.

In order to evaluate one aspect of the safety of zsig37 and its effect on template bleeding time, a pre-clinical model in Macaca fascicularis was used. Time to cessation of bleeding from standardized incisions on the animal's forearm, a procedure previously described by Wu, et al., Blood, 2002, 99:3623-3628, was employed to assess the effect of zsig37 on bleeding.

Methods. Five female and twelve male, Macaca fascicularis weighing 2.3 to 4.6 kg were used as test subjects on this project. The animals were immobilized with ketamine hydrochloride (Phoenix Scientific) administered intramuscularly at approximately 10 mg/kg. Each animal was examined, and the animal determined to be acceptable for the study. The right forearm, abdomen, ventral neck and inguinal area were shaved free of hair. The animal was transported to the surgical suite (University of Washington Regional Primate Research Center), positioned inside a heated air circulation system and placed onto isoflurane (Abbott Laboratories) inhalation anesthesia delivered through positive pressure ventilation via an endotracheal tube. With the animal in a dorsal recumbent position, the right arm was not restrained and an infant blood pressure cuff (LifeSource) placed around the upper arm and inflated to a pressure of 40 mm Hg. On the volar surface of the forearm a standardized depth and length incision was created using the spring-loaded Organon Teknika Simplate II Bleeding Time device (Organon Teknika, Durum, N.C.). The wound was carefully dabbed as blood pooled next to the incision using filter paper or cotton gauze. Care was taken not to touch the incision with the filter paper. The time to cessation of bleeding was determined and recorded for each animal. Bleeding times were determined for each animal prior to any treatment, after low molecular weight heparin (Lovenox™, Rhone Polenc, Inc.) treatment and after zsig37 treatment.

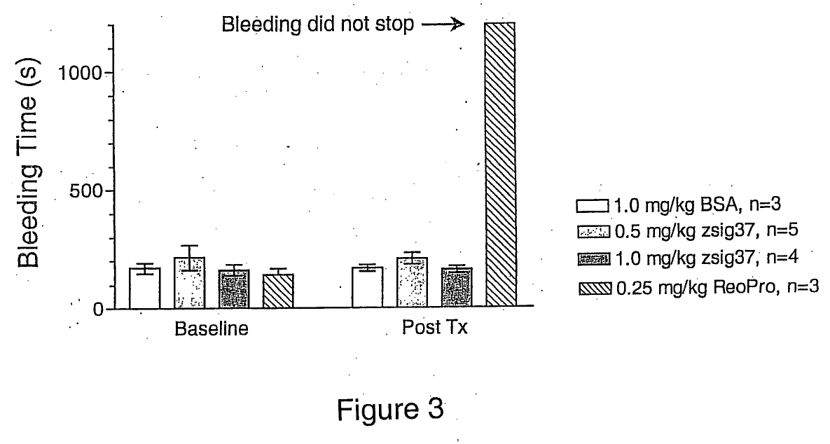

Results. The bleeding times for the Macaca fascicularis treated with 0.5 mg/kg or 1.0 mg/kg of zsig37 was not statistically different from the bleeding times for the control (1.0 mg/kg BSA) group. Thus, zsig37 does not promote bleeding. On the other hand, Macaca fascicularis treated with 0.25 mg/kg ReoPro were unable to stop their bleeding. Consequently, the increase bleeding times of the 0.25 mg/kg ReoPro treated Macaca fascicularis as compared to the control group was statistically different (FIG. 3).

### EXAMPLE 14

### Evaluation of the Effect of zsig37 and Clopidogrel on Blood Loss from an Iliac Artery Catheter Insertion Site in the Rabbit

Introduction. During cardiac and vascular diagnostic and therapeutic procedures, it is common practice to insert a sheath introducer into the femoral artery to be used for catheter access to the vascular system. It is through these sheath introducers that angioplasty catheters, embolectomy catheters, angiogram catheters and stent placement catheters are inserted. The patient may currently be on a platelet inhibitor therapy regimen or may be placed on one prior to, during or after the procedure. Upon completion of the vascular or cardiac procedure, the sheath introducer is removed and compression or vascular closure devices are applied to the catheter exit site to control bleeding. This post procedural bleeding from the catheter exit site may be catastrophic in many cases.

As zsig37 can inhibit platelet activation and aggregation, its effect on bleeding from such a site was studied. An experimental model was employed where insertion and withdrawal of a 22 G angiocath into the iliac artery of rabbit and measurement of blood loss from the exit site. Zsig37 treatment was compared with an inactive buffer as well as clopidogrel (Plavix™, Bristol Myers Squibb), as well as zsig37 co-treated with thrombin.

Methods. Thirteen normal, female New Zealand White rabbits (Western Oregon Rabbit Company) weighing 2.91 to 3.44 kg were used as test subjects for this study. The animals were divided into three groups, with one being the negative control (zsig37 buffer only N=5), another the zsig37 treated group (N=6) and the third group treated with clopidogrel (N=2). The zsig37 buffer (0.9 ml/kg) and the zsig37 (1 mg/kg) were administered IV via an angiocatheter in the marginal ear vein. Clopidogrel (12-15 mg/kg) was administered via an oral gastric tube in two equal doses approximately 19 hours apart. The second clopidogrel dose was administered 45 minutes prior to the study time. Each animal was immobilized with an intramuscular injection of ketamine hydrochloride (Phoenix Scientific) at 50 mg/kg and prepared for the surgical procedure. Hair was shaved from the ventral neck, abdomen and left ear. Via a midline incision in the ventral neck and with blunt dissection, the carotid artery was exposed and a polyurethane catheter (RenaPulse™ High Fidelity Pressure Tubing, BrainTree Scientific, Inc.) implanted for blood pressure measurement (Model BPA, Digimed Corp.). Via a midline incision of the abdomen and an incision over the right iliac, the area surrounding the right iliac artery was cleared of connective tissue and the vessel exposed.

Using a pair of vascular clips, a 2.5 cm section of the right iliac was temporarily clipped and circulation stopped, a 22 G angiocatheter was then inserted into the vessel advanced 1 cm beyond the needle tip and then removed. A pre-weighed-dry 2×2 Nugauze pad was placed directly on the puncture site covered with a second pre-weighed-wetted 3×3 Nugauze pad (Johnson and Johnson) and finger pressure applied as the vessel clips were released. Three pre-weighed-dry 3×3 Nugauze pads (Johnson and Johnson) folded in half were then placed on top of the wetted gauze followed by placement of a 200 gm weight. Finger pressure was released and the site monitored for leakage of blood that was not flowing into the gauze pads. Any leakage of blood around the pads was absorbed into a weighed gauze pad. After five minutes, the weight and the three folded pads were carefully removed and the wetted gauze observed for active bleed through. If bleed through was observed, the pads and weight were replaced and the bleed through reassessed after approximately 90 seconds. This was repeated until bleed through had stopped. The gauze pads were collected and the weight of the absorbed blood determined by subtraction of the gauze weight.

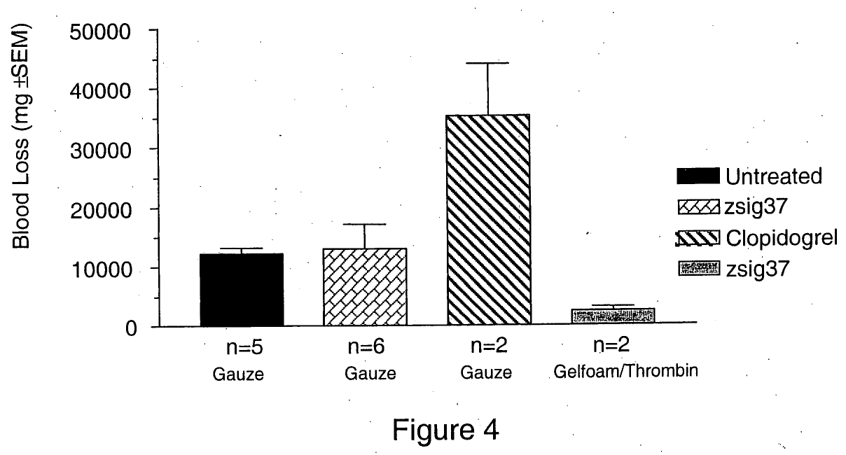

Results. The gauze of zsig37 treated vascular catheter insertion sites did not have a statistically significant difference in accumulation of blood as compared to the control rabbits. The gauze of a zsig37 and thrombin treated site had a statistically significantly lower accumulation of blood as compared to the control rabbits. Zsig37 does not cause adverse bleeding from vascular wounds such as catheter insertion sites, and bleeding can be effective controlled using standard measures, such as gauze or gelfoam/thrombin. On the other hand, clopidogrel treated sites had statistically significant higher accumulation of blood as compared to the control rabbits (FIG. 4).

### EXAMPLE 15

### Inhibition of Platelet Activation by Collagen Related Peptide (CRP)

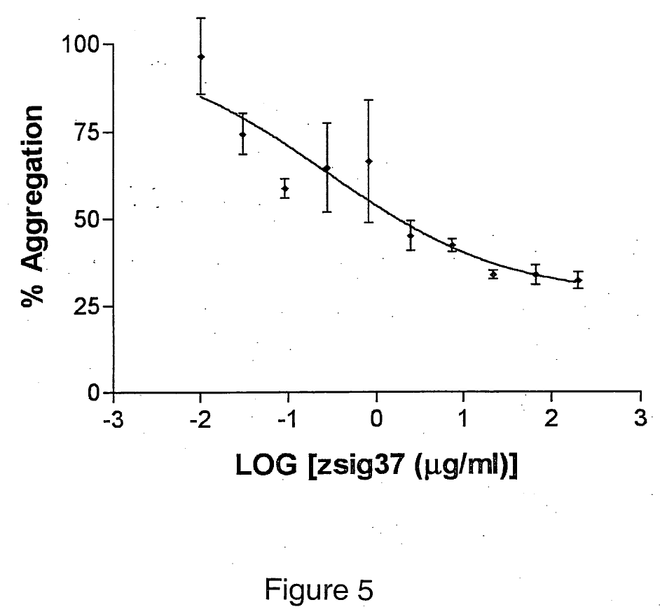

Collagen related peptide (CRP) has been demonstrated to selectively activate the platelet collagen receptor GPVI (Barnes et al., Curr. Opin. Hematol., 5(5):314-320 (1998)). The lysine containing CRP (Ac-GKO-(GPO)₁₀-GKOGV) (SEQ ID NO:15) was synthesized and cross-linked essentially as described by described by Morton (Morton et al., Biochem. J., 306(2):337-344 (Mar. 1, 1995)). The potency of the cross-linked CRP was determined using a using a modified microplate platelet aggregation method as described previously (Bednar B., et al., Thrombosis Research, 77(5):453-463 (1995)). Platelet rich plasma (PRP) was prepared by centrifugation (150 g, 30 min.) from citrated blood obtained from healthy volunteers. Modified Hepes Tyrodes buffer (10 mM Hepes, 137 mM NaCl, 2.7 mM KCL, 0.4 mM NaH₂PO₄, 1.2mM NaHCO₃, 0.1% dextrose and 0.2% BSA fraction V) was used to adjust the platelet concentration to 2.6×10⁸/mL. To determine potency, triplicate wells of 0-20 μg/ml of CRP was mixed with platelets in a 96-well flat bottom plate. As a control, collagen-I norm was assayed in triplicate at a final concentration of 1.25 μg/mL. The plate was agitated on a microplate reader and turbidity was monitored as percent light transmitted at 632 nm. The EC50 for the CRP was determined from 3 assays to be 0.1-0.2 μg/ml. To evaluate the inhibition of CRP activation by zsig37, 100 μl of 5 μg/ml CRP was incubated at 37° C. for 16 hours. The plate was washed three times with 5% BSA/PBS and triplicate wells of 0-200 μg/ml of zsig37 was incubated for 1 hour at room temperature. Platelets were then added and assayed as described. The results from 3 of 6 assays demonstrated inhibition of CRP indicating that zsig37 was blocking interaction with platelet GPVI as shown in FIG. 5.

### EXAMPLE 16

### Platelet Inhibition and Binding Activity of Isolated TNF Domain

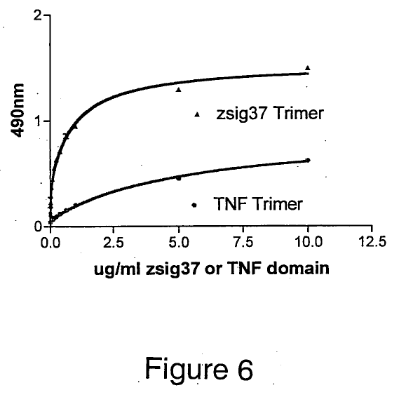

The platelet inhibition and binding activity of zsig37 TNF domain was examined by digesting the collagen-like domain with collagenase. Briefly, 2.25 mg of zsig37 was digested with 0.2 mg of collagenase type IV (Worthington) with 1× complete protease inhibitor at 22° C. overnight. The TNF domain was isolated on a Superdex 200 gel permeation column. The elution profile was consistent with a TNF trimer and was confirmed by non-reducing SDS PAGE. N-terminal sequencing indicated that the new terminus began at G146 and was determined by the Limulus amoebocyte assay to be essentially free of LPS contamination. The isolated TNF domain did not inhibit collagen-induced platelet aggregation at concentrations up to 50 μg/ml. It was also ineffective in the aortic ring relaxation assay at 100 μg/ml. The ELISA collagen binding assay (as described herein) indicated that the isolated TNF domain (kd 2.17 vs. zsig37 trimer kd 0.26) had a greatly reduced affinity for Collagen I (FIG. 6). These data indicate that the TNF domain alone is not sufficient for the described activity of zsig37.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating a vascular disorder in a mammal to maintain or to increase blood flow within the vasculature of the mammal, comprising administering to the mammal a polypeptide comprising a sequence of amino acid residues that is at least 95% identical in amino acid sequence to amino acid residues 26 to 281 of SEQ ID NO:2, wherein the amino acid sequence comprises:
(a) Gly-Xaa-Xaa or Gly-Xaa-Pro repeats forming a collagen domain, wherein Xaa is any amino acid, and
(b) a carboxy-terminal globular portion,
wherein the disorder is selected from the group of acute coronary syndrome, unstable angina, peripheral arterial disease, thrombocytopenia, thrombotic thrombocytopenia purpura, hemolytic uremia syndrome, a vascular disorder associated with blunt trauma, a vascular disorder associated with head trauma, a vascular disorder associated with poly-trauma, deep vein thrombosis, venous thrombosis, and pulmonary embolism.

2. The method of claim 1, wherein the polypeptide comprises amino acid residues 22 to 281 of SEQ ID NO:2.

3. The method of claim 1, wherein any differences between the amino acid sequence of the polypeptide and the corresponding amino acid sequence of SEQ ID NO:2 are due to conservative amino acid substitutions.

4. The method of claim 1, wherein the collagen domain consists of thirteen Gly-Xaa-Xaa repeats and one Gly-Xaa-Pro repeat.

5. The method of claim 1, wherein the globular domain consists of ten beta sheets.

6. The method of claim 5, wherein the beta sheets are associated with amino acid residues corresponding to 147 to 151, 170 to 172, 178 to 181, 191 to 203, 207 to 214, 219 to 225, 227 to 239, 244 to 250, and 269 to 274 of SEQ ID NO:2.

7. The method of claim 1, wherein the polypeptide comprises amino acid residues 1 to 281 of SEQ ID NO:2, or amino acid residues 1 to 281 of SEQ ID NO:5.

8. The method of claim 1 wherein the administration of the polypeptide does not cause bleeding in the mammal.

9. The method of claim 1 wherein the vascular disorder is atherosclerosis.

10. The method of claim 1 wherein the mammal is a human.

11. The method of claim 1 wherein the polypeptide is administered prior to, during, or following an acute vascular injury in the mammal.

12. The method of claim 1, wherein the polypeptide is complexed to a second polypeptide to form an oligomer.

13. The method claim 12, wherein the polypeptides are complexed by intermolecular disulfide bonds.

14. The method of claim 13, wherein the oligomer is a trimer.

15. The method of claim 13, wherein the oligomer is a hexamer.

16. The method of claim 13, wherein the oligomer is an 18mer.

17. A method of treating a vascular disorder in a mammal to maintain or to increase blood flow within the vasculature of the mammal, comprising administering to the mammal a composition comprising a pharmaceutically effective amount of a polypeptide comprising amino acid residues 26 to 281 of SEQ ID NO:2 and a pharmaceutically acceptable carrier, wherein the disorder is selected from the group of acute coronary syndrome, unstable angina, peripheral arterial disease, thrombocytopenia, thrombotic thrombocytopenia purpura, hemolytic uremia syndrome, a vascular disorder associated with blunt trauma, a vascular disorder associated with head trauma, a vascular disorder associated with poly-trauma, deep vein thrombosis, venous thrombosis, and pulmonary embolism.

18. The method of claim 17 wherein the composition does not cause bleeding in the mammal.

19. The method of claim 17 wherein the vascular disorder is atherosclerosis.

20. The method of claim 17 further comprising an additional therapeutic agent.

21. The method of claim 20 wherein the additional therapeutic agent is a tissue plasminogen activator.

22. The method of claim 20 wherein the additional therapeutic agent is a blood coagulation inhibiting factor.

23. The method of claim 17 wherein the additional therapeutic agent is administered before, concomitant with, or after the administration of the polypeptide.


SEQUENCE LISTING (15 sequences)

SEQ ID NO: 1 - DNA, 2769 bp, Homo sapiens
  CDS (171)...(1013)
gaattcgaattcctttgtttccactgggacggaatcggagctctggaggctgggctggccaagcgccccgaaggcccgatgcctgacggctcatgcggcctccttgtttgcagggcctgggcaaaaatttacactgagtcccactcttcgctccagggcccggcaggaagatgggctcccgtggacagggactcttgctggcgtactgcctgctccttgcctttgcctctggcctggtcctgagtcgcgtgccccatgtccagggggaacagcaggagtgggaggggactgaggagctgccgtcccctccggaccatgccgagagggctgaagaacaacatgaaaaatacaggcccagtcaggaccaggggctccctgcttcccggtgcttgcgctgctgtgaccctggtacctccatgtacccggcgaccgccgtgccccagatcaacatcactatcttgaaaggggagaagggtgaccgcggagatcgaggcctccaagggaaatatggcaaaacaggctcagcaggggccaggggccacactggacccaaagggcagaagggctccatgggggcccctggggagcggtgcaagagccactacgccgccttttcggtgggccggaagaagcccatgcacagcaaccactactaccagacggtgatcttcgacacggagttcgtgaacctctacgaccacttcaacatgttcaccggcaagttctactgctacgtgcccggcctctacttcttcagcctcaacgtgcacacctggaaccagaaggagacctacctgcacatcatgaagaacgaggaggaggtggtgatcttgttcgcgcaggtgggcgaccgcagcatcatgcaaagccagagcctgatgctggagctgcgagagcaggaccaggtgtgggtacgcctctacaagggcgaacgtgagaacgccatcttcagcgaggagctggacacctacatcaccttcagtggctacctggtcaagcacgccaccgagccctagctggccggccacctcctttcctctcgccaccttccacccctgcgctgtgctgaccccagggctcagcaccaggctgaccccaccgcctcttccccgatccctggactccgactccctggctttggcattcagtgagacgccctgcacacacagaaagccaaagcgatcggtgctcccagatcccgcagcctctggagagagctgacggcagatgaaatcaccagggcggggcacccgcgagaaccctctgggaccttccgcggccctctctgcacacatcctcaagtgaccccgcacggcgagacgcgggtggcggcagggcgtcccagggtgcggcaccgcggctccagtccttggaaataattaggcaaattctaaaggtctcaaaaggagcaaagtaaaccgtggaggacaaagaaaagggttgttatttttgtctttccagccagcctgctggctcccaagagagaggccttttcagttgagactctgcttaagagaagatccaaagttaaagctctggggtcaggggaggggccgggggcaggaaactacctctggcttaattcttttaagccacgtaggaactttcttgagggataggtggaccctgacatccctgtggccttgcccaagggctctgctggtctttctgagtcacagctgcgaggtgatgggggctggggccccaggcgtcagcctcccagagggacagctgagccccctgccttggctccaggttggtagaagcagccgaagggctcctgacagtggccagggacccctgggtcccccaggcctgcagatgtttctatgaggggcagagctcctggtacatccatgtgtggctctgctccacccctgtgccaccccagagccctggggggtggtctccatgcctgccaccctggcatcggctttctgtgccgcctcccacacaaatcagccccagaaggccccggggctttggcttctgttttttataaaacacctcaagcagcactgcagtctcccatctcctcgtgggctaagcatcaccgcttccacgtgtgttgtgttggttggcagcaaggctgatccagaccccttctgcccccactgccctcatccaggcctctgaccagtagcctgagaggggctttttctaggcttcagagcaggggagagctggaaggggctagaaagctcccgcttgtctgtttctcaggctcctgtgagcctcagtcctgagaccagagtcaagaggaagtacacatcccaatcacccgtgtcaggattcactctcaggagctgggtggcaggagaggcaatagcccctgtggcaattgcaggaccagctggagcagggttgcggtgtctccgcggtgctctcgccctgcccatggccaccccagactctgatctccaggaaccccatagcccctctccacctcaccccatgttgatgcccagggtcactcttgctacccgctgggcccccaaacccccgctgcctctcttccttccccccatcccccacctggttttgactaatcctgcttccctctctgggcctggctgccgggatctggggtccctaagtccctctctttaaagaacttctgcgggtcagactctgaagccgagttgctgtgggcgtgcccggaagcagagcgccacactcgctgcttaagctcccccagctctttccagaaaacattaaactcagaattgtgttttcagcaaaaaaaaaaaaaaaaaaaagggcggccgc

SEQ ID NO: 2 - protein, 281 aa, Homo sapiens
MGSRGQGLLLAYCLLLAFASGLVLSRVPHVQGEQQEWEGTEELPSPPDHAERAEEQHEKYRPSQDQGLPASRCLRCCDPGTSMYPATAVPQINITILKGEKGDRGDRGLQGKYGKTGSAGARGHTGPKGQKGSMGAPGERCKSHYAAFSVGRKKPMHSNHYYQTVIFDTEFVNLYDHFNMFTGKFYCYVPGLYFFSLNVHTWNQKETYLHIMKNEEEVVILFAQVGDRSIMQSQSLMLELREQDQVWVRLYKGERENAIFSEELDTYITFSGYLVKHATEP

SEQ ID NO: 3 - DNA, 843 bp, Artificial Sequence
  This degenerate nucleotide sequence encodes the amino acid sequence of SEQ ID NO2.
atgggnwsnmgnggncarggnytnytnytngcntaytgyytnytnytngcnttygcnwsnggnytngtnytnwsnmgngtnccncaygtncarggngarcarcargartgggarggnacngargarytnccnwsnccnccngaycaygcngarmgngcngargarcarcaygaraartaymgnccnwsncargaycarggnytnccngcnwsnmgntgyytnmgntgytgygayccnggnacnwsnatgtayccngcnacngcngtnccncarathaayathacnathytnaarggngaraarggngaymgnggngaymgnggnytncarggnaartayggnaaracnggnwsngcnggngcnmgnggncayacnggnccnaarggncaraarggnwsnatgggngcnccnggngarmgntgyaarwsncaytaygcngcnttywsngtnggnmgnaaraarccnatgcaywsnaaycaytaytaycaracngtnathttygayacngarttygtnaayytntaygaycayttyaayatgttyacnggnaarttytaytgytaygtnccnggnytntayttyttywsnytnaaygtncayacntggaaycaraargaracntayytncayathatgaaraaygargargargtngtnathytnttygcncargtnggngaymgnwsnathatgcarwsncarwsnytnatgytngarytnmgngarcargaycargtntgggtnmgnytntayaarggngarmgngaraaygcnathttywsngargarytngayacntayathacnttywsnggntayytngtnaarcaygcnacngarccn

SEQ ID NO: 4 - DNA, 2559 bp, Mus musculus
  CDS (70)...(912)
gaattcggatcctggaagagatgggattgttataggcggaaagagagaaacccagagaagtccaggaagatgggctcctgtgcacagggattcatgctgggatgctgcctgctgctggccatcacctggggccccatcctgagccttgtgccacgcgttcaggaggaacaacaggagtgggaagagacagaggagctgccatctcctctggatcctgtgacaaggcctgaagaaacacgagagaagtatagccctcgccagggtgaggacctccccacttctcggtgctaccgatgctgtgaccccagcacacctgtataccagacaattcctccaccccagatcaacatcaccatcctgaaaggcgagaaaggtgaccgaggggatcgaggcctccaggggaagtacggcaaaataggttctacaggtcccaggggccatgttggccccaaagggcagaagggatccattggagcccctgggaaccactgcaagagccagtacgcagccttctccgtgggccggaagaaggctttgcacagcaacgactacttccagcccgtggtcttcgacacggagtttgtgaacctctacaaacacttcaatatgttcactgggaagttctactgctatgtgccgggcatctacttcttcagcctcaacgtgcacacttggaaccagaaggagacgtacctgcacatcatgaagaacgaggaggaggtggtgatcctgtatgcgcaggtgagcgaccgcagcatcatgcagagtcagagcctgatgatggagctgcgggaggaggatgaggtctgggtgcgtctcttcaagggcgagcgtgagaacgccattttcagtgacgagttcgacacctacatcaccttcagtggctacctggtcaagccagcctctgagccctagtggacactcctgtggagcttttgtggactgctgacctccttgcctggcaccctgacctatccctgcattctacagacactggagtcctgccccgggctgaccccattttctctctgctccatcctggcttccttggccttggcttccaaagttttggcttttgacaagatgcccttggccactgggaatcccaaaggatggtgcgatcccagatctggctgctactctaagcagagagctgccggcagatgaaatcattgggcggggagcctgtgaggatattggggggcctccagctccttctgtgtacacagccttagacgaccctgtgctgtgttgtcccgtggccacagggtgttccagagcacagcccctgtgtgttcccattcctggacacaagtaagcaaatatcatgggtttcttaggaacgaagtcaagcagaaaagagaaagaaaggtggtgttagttttggctttccagccagctggaaggagggatggggagagagagagagagagagctatttgtattggggaaactgaggcataggaaaaacatgaatggcaacagagtagctgcagtttgtgggtttggaaaccacatctgacttaactctagatcacatatgagctttcctggggacagcaggactgacctccgagctctgttgacatgctatagccttgcccaggggctggtcaatctttctgagccacactagtaaaagggttggaggagaacagcaagtgccccctgtggttggctctgggctggtggcagcatcctgcttgccccaactcacaggatcctgacagcagctgggaacctcagggactcctgcagctttctctgtaagaaataaagctcctactatgtcccagtacctctctgctctgctccacttccccagtcactctggaccccagggtgggagggctctcttgcctgttgggacatcagttccccttcctccttcttggtgaattaaccatggaaggaccagggctcggatttgggttcccaaactgcccttcaccatccctagtgtcctgcttccttcccagttcagcatcctgtctgggaacttgatactttaacctgctagagcggatgagtctgatagacctgcccagccctgacacagccctagtcagcttatggacacgtgagagggacttcctttgagacccagagctggggtagagctataaaaatctacctattcccgggtcaaccccaagtggtagaagaggacacaggctatcccgccctagctcagactcagggaaggcctcaggcctgattgtctgactgcagagagcctgtgttctttccccatctcaccccgtgttgatccccagggcctgggccactggatatctgctttgtgccaactaggccttgcttgctgcttcctggtggcccttggttaggatccctctcttttccttctggagctcaatgtacgtatatgccacctccgaaggggcttctgctggtcagactctccaagccacttccatgggtgtgcctacagcagaggctgctgcctcctgtgctctaccctgctctttccagaaaacattaaacttgccatggcgattcacagcaaaaaaaaaaaaaaaaaaaaaaaaagggcggccg

SEQ ID NO: 5 - protein, 281 aa, Mus musculus
MGSCAQGFMLGCCLLLAITWGPILSLVPRVQEEQQEWEETEELPSPLDPVTRPEETREKYSPRQGEDLPTSRCYRCCDPSTPVYQTIPPPQINITILKGEKGDRGDRGLQGKYGKIGSTGPRGHVGPKGQKGSIGAPGNHCKSQYAAFSVGRKKALHSNDYFQPVVFDTEFVNLYKHFNMFTGKFYCYVPGIYFFSLNVHTWNQKETYLHIMKNEEEVVILYAQVSDRSIMQSQSLMMELREEDEVWVRLFKGERENAIFSDEFDTYITFSGYLVKPASEP

SEQ ID NO: 6 - DNA, 843 bp, Artificial Sequence
  This degenerate nucleotide sequence encodes the amino acid sequence of SEQ ID NO5.
atgggnwsntgygcncarggnttyatgytnggntgytgyytnytnytngcnathacntggggnccnathytnwsnytngtnccnmgngtncargargarcarcargartgggargaracngargarytnccnwsnccnytngayccngtnacnmgnccngargaracnmgngaraartaywsnccnmgncarggngargayytnccnacnwsnmgntgytaymgntgytgygayccnwsnacnccngtntaycaracnathccnccnccncarathaayathacnathytnaarggngaraarggngaymgnggngaymgnggnytncarggnaartayggnaarathggnwsnacnggnccnmgnggncaygtnggnccnaarggncaraarggnwsnathggngcnccnggnaaycaytgyaarwsncartaygcngcnttywsngtnggnmgnaaraargcnytncaywsnaaygaytayttycarccngtngtnttygayacngarttygtnaayytntayaarcayttyaayatgttyacnggnaarttytaytgytaygtnccnggnathtayttyttywsnytnaaygtncayacntggaaycaraargaracntayytncayathatgaaraaygargargargtngtnathytntaygcncargtnwsngaymgnwsnathatgcarwsncarwsnytnatgatggarytnmgngargargaygargtntgggtnmgnytnttyaarggngarmgngaraaygcnathttywsngaygarttygayacntayathacnttywsnggntayytngtnaarccngcnwsngarccn

SEQ ID NO: 7 - protein, 10 aa, Artificial Sequence
  Fragment of zacrp3.
PDCSKCCHGD

SEQ ID NO: 8 - protein, 10 aa, Artificial Sequence
  Fragment of zacrp5.
RPCVHCCRPA

SEQ ID NO: 9 - protein, 10 aa, Artificial Sequence
  Fragment of zacrp6.
SGCQRCCDSE

SEQ ID NO: 10 - protein, 75 aa, Artificial Sequence
  Fragment of zacrp3.
PDCSKCCHGDYSFRGYQGPPGPPGPPGIPGNHGNNGNNGATGHEGAKGEKGDKGDLGPRGERGQHGPKGEKGYPG

SEQ ID NO: 11 - protein, 88 aa, Artificial Sequence
  Fragment of zacrp5.
RPCVHCCRPAWPPGPYARVSDRDLWRGDLWRGLPRVRPTINIEILKGEKGEAGVRGRAGRSGKEGPPGARGLQGRRGQKGQVGPPGAA

SEQ ID NO: 12 - protein, 83 aa, Artificial Sequence
  Fragment of zacrp6.
SGCQRCCDSEDPLDPAHVSSASSSGRPHALPEIRPYINITILKGDKGDPGPMGLPGYMGREGPQGEPGPQGSKGDKGEMGSPG

SEQ ID NO: 13 - DNA, 24 bp, Artificial Sequence
  PCR primer.
tcccctttcaagatagtgatgttg

SEQ ID NO: 14 - DNA, 23 bp, Artificial Sequence
  PCR primer.
catgaaaaatacaggcccagtca

SEQ ID NO: 15 - protein, 38 aa, Artificial Sequence
  Collagen Related Peptide
XKXGPXGPXGPXGPXGPXGPXGPXGPXGPXGPXGKXGV